(19) United States
(12) Patent Application Publication
(10) Pub. No.: US 2021/0040061 A1
(43) Pub. Date: Feb. 11, 2021

(54) COMPOUND, POLYMER, AND ORGANIC MATERIAL

(71) Applicant: Sony Corporation, Tokyo (JP)

(72) Inventors: Kenshiro KAWASAKI, Kanagawa (JP); Hisaya HARA, Kanagawa (JP)

(21) Appl. No.: 16/976,301

(22) PCT Filed: Feb. 26, 2019
(86) PCT No.: PCT/JP2019/007330

§ 371 Date: Aug. 27, 2020

(30) Foreign Application Priority Data
Feb. 27, 2018 (JP) 2018-033835

(51) Int. Cl. C07D 333/50; C08F 20/38; G02B 1/04; G03H 1/02

(52) U.S. Cl. CPC C07D 333/50; C08F 20/38; G03H 2001/0264; G03H 1/02; G02B 1/041

(57) ABSTRACT

Providing a compound that can realize an organic material with an enhanced function.

Provided is a compound represented by the following general formula (1).

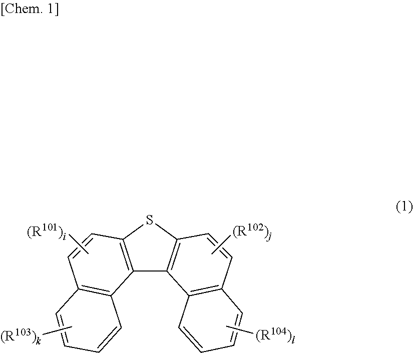

(In the general formula (1), R¹⁰¹ to R¹⁰⁴ are each independently a univalent substituent group represented by the following general formula (2-1), i to 1 are each independently an integer of 0 or 1, provided that i to 1 are not simultaneously 0.)

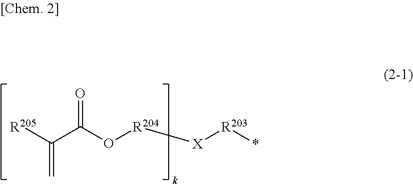

(In the general formula (2-1), R²⁰³ and R²⁰⁴ are each independently a single bond or a straight chain or branched substituted or unsubstituted alkylene group represented by CₙH₂ₙ (n is an integer of equal to or greater than 1), R²⁰⁵ is hydrogen or a straight chain or branched substituted or unsubstituted alkyl group represented by CₙH₂ₙ₊₁ (n is an integer of equal to or greater than 1). Represented by k is an integer of equal to or greater than 1, and X is a bivalent or more-valent aromatic group. If carbon not bonded to R²⁰³ and R²⁰⁴ is present in the bivalent or more-valent aromatic group, the carbon is unsubstituted or has at least one substituent group. In addition, a part for bonding to R²⁰³ and at least one part for bonding to R²⁰⁴, possessed by the bivalent or more-valent aromatic group, may be any bondable carbon in the aromatic group. Represented by * in R¹⁰¹ to R¹⁰² is a part for bonding with carbon that is bondable in a benzene ring condensed with a thiophene ring in the general formula (1). Represented by * in R¹⁰³ to R¹⁰⁴ is a part for bonding with carbon that is bondable in the benzene ring not condensed with the thiophene ring in the general formula (1).)

### TECHNICAL FIELD

The present technology relates to a compound, a polymer, and an organic material.

### BACKGROUND ART

High-functional organic materials are excellent in the degree of freedom in design and in shock resistance and are light in weight, as compared to inorganic materials. Therefore, there have been vigorous investigations for application of the high-functional organic materials to optical materials such as organic thin film, organic lens, and hologram.

For example, there has been proposed a curable composition having curing shrinkage resistance, containing a polymerizable compound in which a polymerizable substituent group is introduced to a 1,1′-binaphthyl skeleton with 2,2′-positions connected to each other by a bivalent substituent group or atom and a polymerization initiator capable of putting the polymerizable substituent group into a polymerization reaction (see PTL 1).

In addition, for example, a refractive index enhancing agent containing a compound having a dinaphthothiophene skeleton has been proposed (see PTL 2). Further, for example, a method of imparting a refractive index to an article by use of a compound having a dibenzothiophene skeleton has been proposed (see PTL 3).

### CITATION LIST

### Patent Literature

### [PTL 1]

JP 2012-136576A

### [PTL 2]

JP 2011-178985A

### [PTL 3]

JP 2011-162584A

### SUMMARY

### Technical Problem

However, according to the technologies proposed by PTL 1 to PTL 3, it may be impossible to realize further enhancement of functions of organic materials.

The present technology has been made in consideration of the above-mentioned circumstances. It is a principal object of the present technology to provide a compound and a polymer which are able to further enhance functions of an organic material, and a highly functional organic material.

### Solution to Problem

The present inventors, as a result of their extensive and intensive research for solving the above-mentioned problem, have surprisingly succeeded in the development of a compound and a polymer capable of realizing enhancement of functions and a highly functional organic material and have completed the present technology.

The present technology provides a compound represented by the following general formula (1).

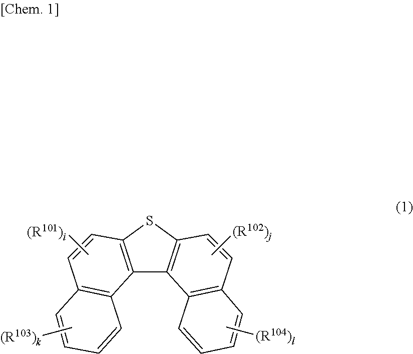

(In the general formula (1), R¹⁰¹ to R¹⁰⁴ are each independently a univalent substituent group represented by the following general formula (2-1), and i to 1 are each independently an integer of 0 or 1, provided that i to 1 are not simultaneously 0.)

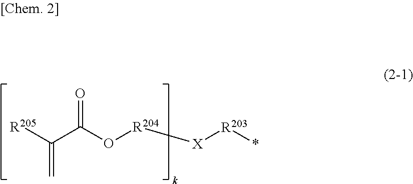

(In the general formula (2-1), R²⁰³ and R²⁰⁴ are each independently a single bond or a straight chain or branched substituted or unsubstituted alkylene group represented by CₙH₂ₙ (n is an integer of equal to or greater than 1), and R²⁰¹ is hydrogen or a straight chain or branched substituted or unsubstituted alkyl group represented by CₙH₂ₙ₊₁ (n is an integer of equal to or greater than 1). Represented by k is an integer of equal to or greater than 1, and X is a bivalent or more-valent aromatic group. If carbon not bonded to R²⁰³ and R²⁰⁴ is present in the bivalent or more-valent aromatic group, the carbon is unsubstituted or has at least one substituent group. In addition, a part for bonding to R²⁰³ and at least one part for bonding to R²⁰⁴, possessed by the bivalent or more-valent aromatic group, may be any bondable carbon in the aromatic group. Represented by * in R¹⁰¹ to R¹⁰² is a part for bonding with carbon that is bondable in a benzene ring condensed with a thiophene ring in the general formula (1). Represented by * in R¹⁰³ to R¹⁰⁴ is a part for bonding with carbon that is bondable in the benzene ring not condensed with the thiophene ring in the general formula (1).)

In the compound according to the present technology, at least one carbon atom of at least one carbon skeleton, of carbon skeletons constituting the alkylene groups of R²⁰³ and R²⁰⁴ and the alkyl group of R²⁰⁵, may be substituted by a hetero atom.

In the compound according to the present technology, at least one hydrogen atom, of hydrogen atoms constituting the alkylene group of R²⁰³, hydrogen atoms constituting the alkylene group of R²⁰⁴, and hydrogen atoms constituting the alkyl group of R²⁰⁵, may be substituted by a halogen atom.

In the compound according to the present technology, R²⁰³ and R²⁰⁴ may be single bonds or straight chain or branched substituted or unsubstituted alkylene groups represented by CₙH₂ₙ (n is an integer of 1≤n≤10), further, R²⁰⁵ may be hydrogen or a straight chain or branched substituted or unsubstituted alkyl group represented by CₙH₂ₙ₊₁ (n is an integer of 1≤n≤10), and, in that case, at least one carbon atom of at least one carbon skeleton, of carbon skeletons constituting the alkylene groups of R²⁰³ and R²⁰⁴ and the alkyl group of R²⁰⁵, may be substituted by a hetero atom, and at least one hydrogen atom, of the hydrogen atoms constituting the alkylene group of R²⁰³, the hydrogen atoms constituting the alkylene group of R²⁰⁴, and the hydrogen atoms constituting the alkyl group of R²⁰⁵, may be substituted by a halogen atom.

X may be a bivalent or more-valent aromatic group represented by the following general formulas (3-1) to (3-8).

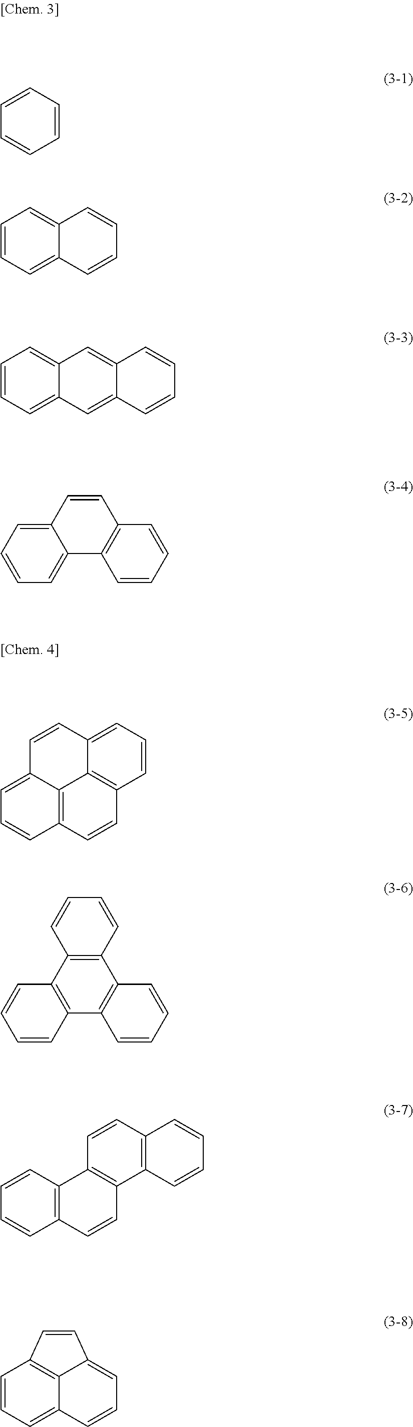

In the compound according to the present technology, k may be 1, and X may be a bivalent aromatic group.

The bivalent aromatic group may be a monocyclic arylene group, and two parts for bonding to R²⁰³ and R²⁰⁴, possessed by the monocyclic arylene group, may be in a relation of ortho positions, meta positions, or para positions.

The bivalent aromatic group may be a polycyclic arylene group, and two parts for bonding to R²⁰³ and R²⁰⁴, possessed by the polycyclic arylene group, may be any two bondable carbon atoms in the polycyclic arylene group.

In the compound according to the present technology, k may be 2, and X may be a trivalent aromatic group.

The trivalent aromatic group may be a monocyclic trivalent aromatic group, and two parts for bonding to R²⁰⁴, possessed by the monocyclic trivalent aromatic group, may be in a relation of ortho positions, meta positions, or para positions.

In the compound according to the present technology, at least one of R¹⁰¹ or R¹⁰² may be adjacent to a carbon atom adjacent to a sulfur atom in the general formula (1) and may be bonded to bondable carbon in the benzene ring condensed with the thiophene ring in the general formula (1).

In the compound according to the present technology, at least one of R¹⁰¹ or R¹⁰² may be adjacent to a carbon atom adjacent to the sulfur atom in the general formula (1), and may be bonded to bondable carbon in the benzene ring condensed with the thiophene ring in the general formula (1).

In addition, the present technology provides an organic material including the compound according to the present technology, and the organic material including the compound, according to the present technology, may be an organic thin film, an organic lens, or a hologram, or may be an organic thin film composition, an organic lens composition, or a hologram recording photosensitive composition.

Further, the present technology provides a polymer obtained by polymerizing the compound according to the present technology.

Furthermore, the present technology provides an organic material including the polymer according to the present technology. The organic material including the polymer, according to the present technology, may be an organic thin film, an organic lens, or a hologram, or may be an organic thin film composition, an organic lens composition, or a hologram recording photosensitive composition.

According to the present technology, enhancement of functions of an organic material can be achieved. Note that the effects described here are not limitative, and any of the effects described herein may be mentioned.

### DESCRIPTION OF EMBODIMENTS

Preferred embodiments for carrying out the present technology will be described below. The embodiments described below are examples of typical embodiments of the present technology, and the scope of the present technology is not to be construed as limited by such embodiments.

Note that the description will be made in the following order.

1. Outline of the present technology
2. First embodiment (Examples of compound)
3. Second embodiment (Examples of polymer)
4. Third embodiment (Examples of organic material)

4-1. Organic thin film and organic thin film composition

4-2. Organic lens and organic lens composition

4-3. Hologram recording photosensitive composition and hologram

5. Fourth embodiment (Example of image display device)
6. Fifth embodiment (Example of optical part)
7. Sixth embodiment (Example of optical device)

### 1. Outline of the Present Technology

First, an outline of the present technology will be described.

The present technology relates to a compound, a polymer, and an organic material.

For example, an organic compound and a polymer having a high refractivity property are considered as a high refractive index material when the refractive index exceeds 1.5. Production of an organic polymer having such a high refractive index can be achieved, for example, by use of a polymerizable monomer in which a polymerizable substituent group is introduced to dinaphthothiophene having a refractive index of 1.8. However, in the case of applying these compounds to optical materials, the following facts are present.
The compounds are low in solubility in organic solvents, and it is difficult to form films thereof using solutions.
The compounds are poor in compatibility with resins, and it is impossible to enlarge the compound concentrations in mixtures.
Some of the compounds are colored and are not suited to application to transparent thin films and lenses.

For example, it is possible to synthesize dinaphthothiophene derivatives having various polymerizable substituent groups and to perform measurement of refractive index and transparency of the derivatives. One of advantages of using high refractive index organic compounds and polymers is that the compounds can be dissolved in organic solvents and thin films of the compounds can easily be produced using a coating step. However, it has been confirmed that as the refractive index of a compound is higher, the solubility of the compound in organic solvents tends to be lowered, and this similarly applies to dinaphthothiophene derivatives. In order to utilize a compound having a high refractive index by dissolving the compound in an organic solvent, it is desirable for the compound to have both a refractive index of equal to or more than 1.7 and a solubility of equal to or more than 20 wt %. In addition to the fact that the degree of freedom in film thickness at the time of producing a film by coating is enhanced as the solubility is higher, the concentration of the high refractive index compound can be enhanced when using the compound through compatibility with other organic compound, and, as a result, average refractive index of the mixture as a whole can be enhanced.

Taking the foregoing into consideration, there has not been found out a dinaphthothiophene derivative having high functionality, for example, simultaneously having a high refractive index, high solubility, and high transparency. The present inventors, as a result of their extensive and intensive investigations, have succeeded in enhancing solubility while maintaining a high refractive index by introducing a polymerizable substituent group having a specific structure to dinaphthothiophene.

There is a technology of introducing an alkyl chain in order to enhance solubility of an organic compound poor in solubility. For example, pentacene can be utilized as an organic semiconductor, but solubility of pentacene in organic solvents is extremely poor, and thus formation of a thin film of pentacene by vapor deposition is a mainstream. There is an example of introducing an alkyl group to the pentacene skeleton for enhancing solubility, thereby enhancing the solubility of pentacene in general purpose organic solvents such as toluene.

Although there is thus an example of introducing an alkyl group, particularly a long chain alkyl group, to an organic compound which is poorly soluble to thereby improve the solubility of the compound in organic solvents, it is easily supposed that the introduction of an alkyl group, particularly a long chain alkyl group, causes the distance between fundamental skeletons to be enlarged.

In the case where an alkyl group is introduced into a fundamental skeleton of a high refractive index organic compound, the solubility of the compound can be enhanced, but, since the refractive index of the alkyl group itself is low and the distance between the fundamental skeletons having the high refractive index is elongated, lowering in the refractive index would arise, and it is difficult to maintain the high refractive index (a refractive index of equal to or more than 1.7). Therefore, it is very difficult to realize a high solubility while maintaining the refractive index of a high refractive index compound.

In consideration of the above-mentioned circumstances, the present inventors have found out that, by introducing a substituent group having a specific structure, high solubility and high refractive index and high transparency can be realized even in regard of a compound having a dinaphthothiophene skeleton.

### 2. First Embodiment (Examples of Compound)

A compound of a first embodiment (examples of compound) according to the present technology is a compound represented by the following general formula (1).

The compound of the first embodiment according to the present technology is able to realize further enhancement of functions of organic materials. In other words, the compound of the first embodiment according to the present technology simultaneously has high solubility and high transparency and high refractive index and is thereby able to realize further enhancement of functions of organic materials. The compound of the first embodiment according to the present technology is able, by introducing a substituent group including an alkyl acrylate and a monocyclic or polycyclic aromatic structure to dinaphthothiophene, to enhance solubility while maintaining a refractive index intrinsic of the dinaphthothiophene mother skeleton.

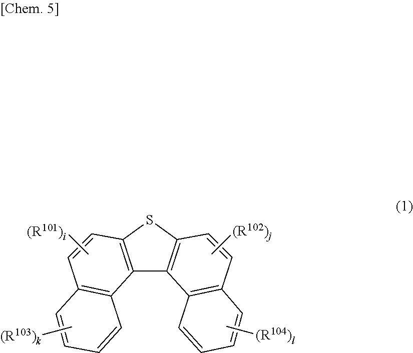

(In the general formula (1), R¹⁰¹ to R¹⁰⁴ are each independently a univalent substituent group represented by the following general formula (2-1), and i to 1 are each independently an integer of 0 or 1, provided that i to 1 are not simultaneously 0.)

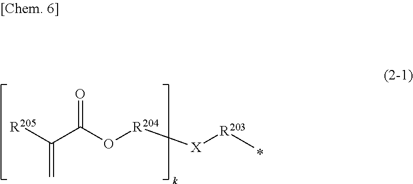

In the general formula (2-1), R²⁰³ and R²⁰⁴ are each independently a single bond or a straight chain or branched substituted or unsubstituted alkylene group represented by CₙH₂ₙ (n is an integer of equal to or greater than 1), and R²⁰⁵ is hydrogen or a straight chain or branched substituted or unsubstituted alkyl group represented by CₙH₂ₙ₊₁ (n is an integer of equal to or greater than 1). Represented by k is an integer of equal to or greater than 1, and X is a bivalent or more-valent aromatic group. If carbon not bonded to R²⁰³ and R²⁰⁴ is present in the bivalent or more-valent aromatic group, the carbon is unsubstituted or has at least one substituent group. In addition, a part for bonding to R²⁰³ and at least one part for bonding to R²⁰⁴, possessed by the bivalent or more-valent aromatic group, may be any bondable carbon in the aromatic group. Represented by * in R¹⁰¹ to R¹⁰² is a part for bonding with carbon that is bondable in a benzene ring condensed with the thiophene ring in the general formula (1). Represented by * in R¹⁰³ to R¹⁰⁴ is a part for bonding with carbon that is bondable in the benzene ring not condensed with the thiophene ring in the general formula (1).

At least one carbon atom possessed by respective carbon skeletons of R²⁰³ to R²⁰⁵ may be substituted by a hetero atom (for example, O, S, N, P), and at least one hydrogen atom possessed by R²⁰³ to R²⁰⁵ may be substituted by a halogen atom (F, Cl, Br, I).

R²⁰³ in the general formula (2) is preferably a single bond or a straight chain or branched substituted or unsubstituted alkylene group represented by CₙH₂ₙ (n is an integer of 1≤n≤10), and more preferably a single bond or a straight chain or branched substituted or unsubstituted alkylene group represented by CₙH₂ₙ (n is an integer of 1≤n≤3). In the case where R²⁰³ is a straight chain or branched alkylene group of 1 to 10 carbon atoms, examples thereof include methylene group, ethylene group, propylene group, isopropylene group, butylene group, and isobutylene group. At least one carbon atom possessed by the carbon skeleton of the straight chain or branched alkylene group of 1 to 10 carbon atoms may be substituted by a hetero atom (for example, O, S, N, P). In addition, at least one hydrogen atom possessed by the straight chain or branched alkylene group of 1 to 10 carbon atoms may be substituted by a halogen atom (F, Cl, Br, I).

R²⁰⁴ in the general formula (2) is preferably a single bond or a straight chain or branched substituted or unsubstituted alkylene group represented by CₙH₂ₙ (n is an integer of 1≤n≤10). In the case where R²⁰⁴ is a straight chain or branched alkylene group of 1 to 10 carbon atoms, examples thereof include methylene group, ethylene group, propylene group, isopropylene group, butylene group, and isobutylene group. At least one carbon atom possessed by the carbon skeleton of the straight chain or branched alkylene group of 1 to 10 carbon atoms may be substituted by a hetero atom (for example, O, S, N, P). Besides, at least one hydrogen atom possessed by the straight chain or branched alkylene group of 1 to 10 carbon atoms may be substituted by a halogen atom (F, Cl, Br, I).

R²⁰⁵ in the general formula (2) is preferably hydrogen or a straight chain or branched substituted or unsubstituted alkyl group represented by CₙH₂ₙ₊₁ (n is an integer of 0≤n≤10). In the case where R²⁰¹ is a straight chain or branched alkyl group of 1 to 10 carbon atoms, examples thereof include methyl group, ethyl group, propyl group, isopropyl group, butyl group, and isobutyl group. At least one carbon atom possessed by the carbon skeleton of the straight chain or branched alkyl group of 1 to 10 carbon atoms may be substituted by a hetero atom (for example, O, S, N, P). In addition, at least one hydrogen atom possessed by the straight chain or branched alkyl group of 1 to 10 carbon atoms may be substituted by a halogen atom (F, Cl, Br, I).

X in the general formula (2) is preferably a bivalent or more-valent aromatic group represented by the following chemical formulas (3-1) to (3-8).

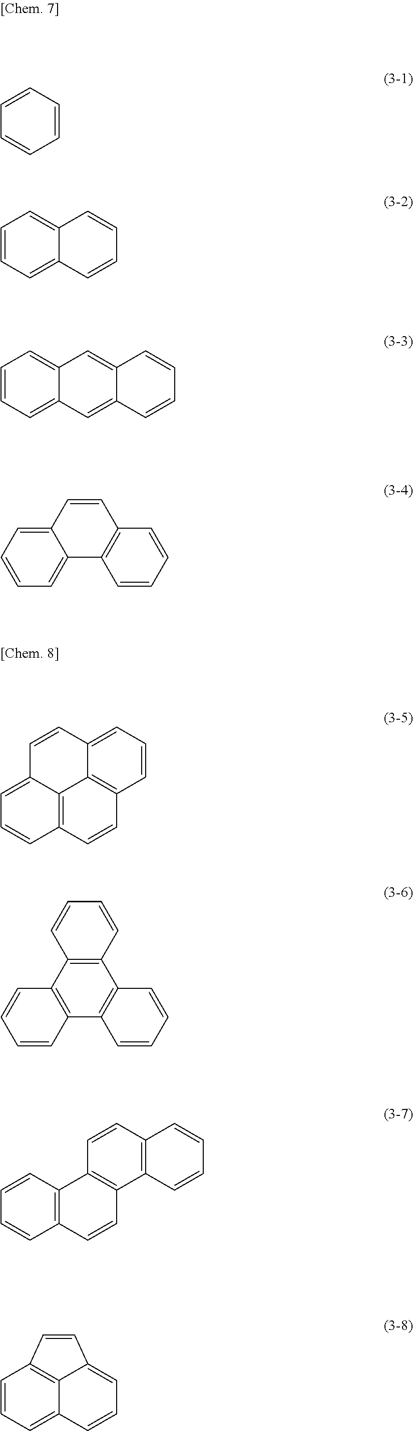

Where the bivalent or more-valent aromatic group has at least one substituent group, the substituent group is preferably a straight chain or branched alkyl group of 1 to 10 carbon atoms, an aromatic group, or a halogen atom. Examples of the straight chain or branched alkyl group of 1 to 10 carbon atoms include methyl group, ethyl group, propyl group, isopropyl group, butyl group, and isobutyl group. In addition, at least one carbon atom possessed by the carbon skeleton of the straight chain or branched alkyl group of 1 to 10 carbon atoms may be substituted by a hetero atom (for example, O, S, N, P). Besides, at least one hydrogen atom possessed by the straight chain or branched alkyl group of 1 to 10 carbon atoms may be substituted by a halogen atom (F, Cl, Br, I). In addition, the aromatic group is preferably a univalent or more-valent aromatic group represented by (3-1) to (3-8) above, which may be unsubstituted or may have at least one substituent group. When the univalent or more-valent aromatic group has at least one substituent group, the substituent group, like the substituent group of X, is preferably a straight chain or branched alkyl group of 1 to 10 carbon atoms (At least one carbon atom in the carbon skeleton of the alkyl group may be substituted by a hetero atom (for example, O, S, N, P). Besides, at least one hydrogen atom possessed by the alkyl group may be substituted by a halogen atom (F, Cl, Br, I).), an aromatic group, or a halogen atom.

When the aromatic group is a bivalent aromatic group (k=1), the bivalent aromatic group may be a monocyclic arylene group, and the two parts for bonding to R²⁰³ and R²⁰⁴, possessed by the monocyclic arylene group, may be in the relation of ortho positions, meta positions, or para positions. In addition, the bivalent aromatic group may be a polycyclic arylene group, and the two parts for bonding to R²⁰³ and R²⁰⁴, possessed by the polycyclic arylene group, may be any two bondable carbon atoms in the polycyclic arylene group.

When the aromatic group is a trivalent aromatic group (k=2), the trivalent aromatic group may be a monocyclic trivalent aromatic group, and the two parts for bonding to R²⁰⁴, possessed by the monocyclic trivalent aromatic group, may be in the relation of ortho positions, meta positions, or para positions. In addition, the trivalent aromatic group may be a monocyclic trivalent aromatic group, and the part for bonding to R²⁰³ and one part of the two parts for bonding to the two R²⁰⁴ groups, possessed by the monocyclic trivalent aromatic group, may be in the relation of ortho positions, meta positions, or para positions.

Further, the trivalent aromatic group may be a polycyclic trivalent aromatic group, and the part for bonding to R²⁰³ and one part of the two parts for bonding to the two R²⁰⁴ groups, possessed by the polycyclic trivalent aromatic group, may be any two bondable carbon atoms in the polycyclic trivalent aromatic group. Furthermore, the trivalent aromatic group may be a polycyclic trivalent aromatic group, and the two parts for bonding to R²⁰⁴, possessed by the polycyclic trivalent aromatic group, may be any two bondable carbon atoms in the polycyclic trivalent aromatic group.

The chemical structures of compounds 4-1 to 4-9 and 11-1 mentioned as preferred monofunctional examples of the compound represented by the above general formula (1) are as follows.

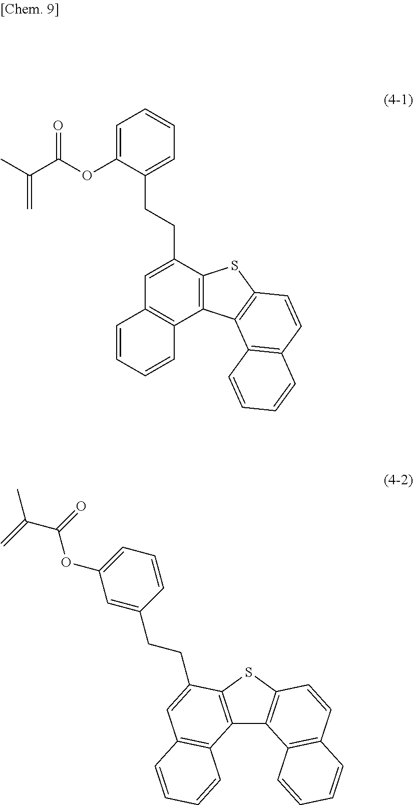

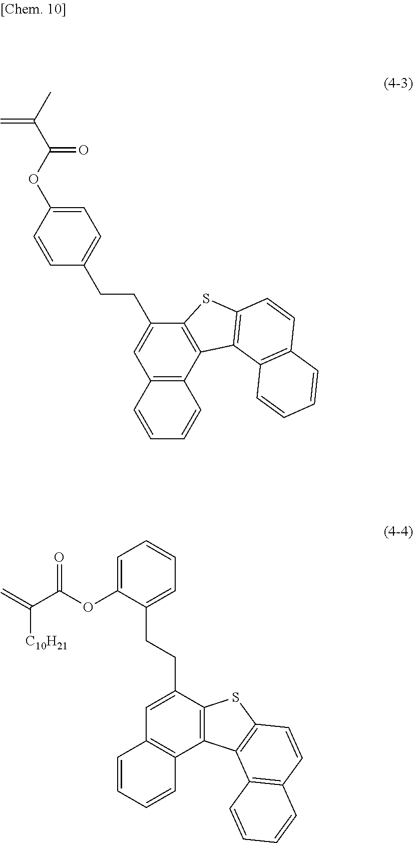

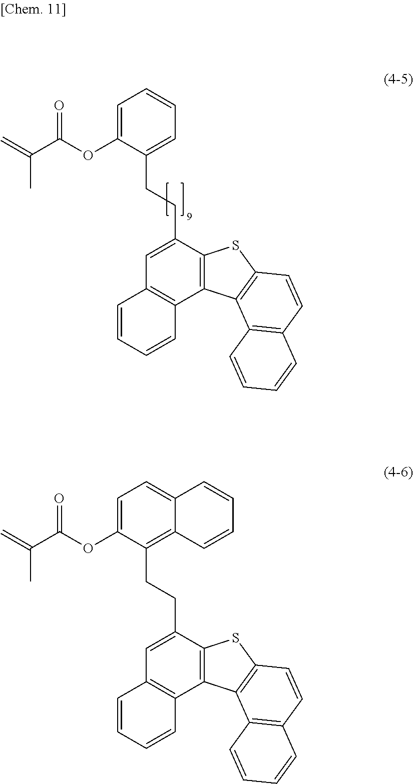

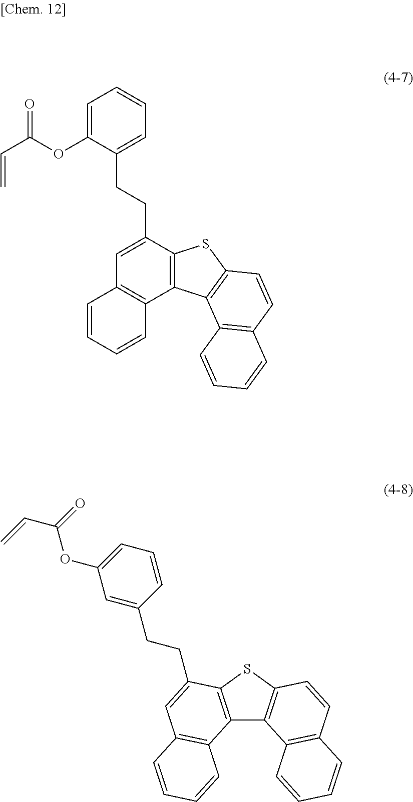

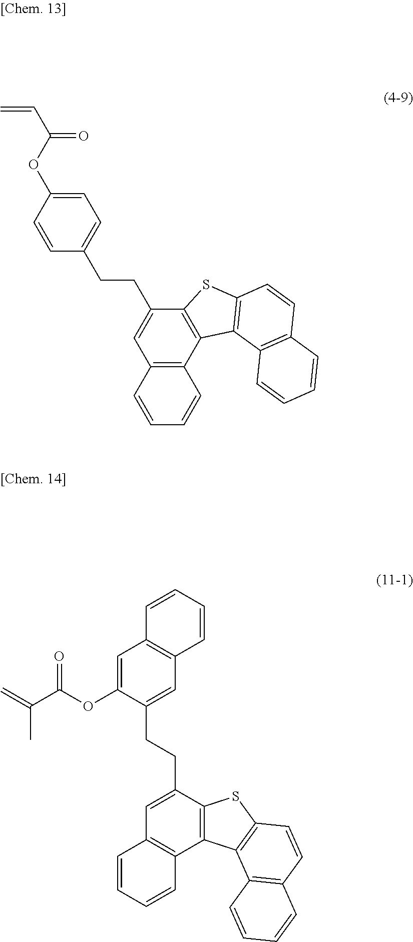

The chemical structural formulas of compounds 5-1 to 5-9 and 10-1 to 10-2 mentioned as preferred bifunctional examples of the compound represented by the above general formula (1) are as follows.

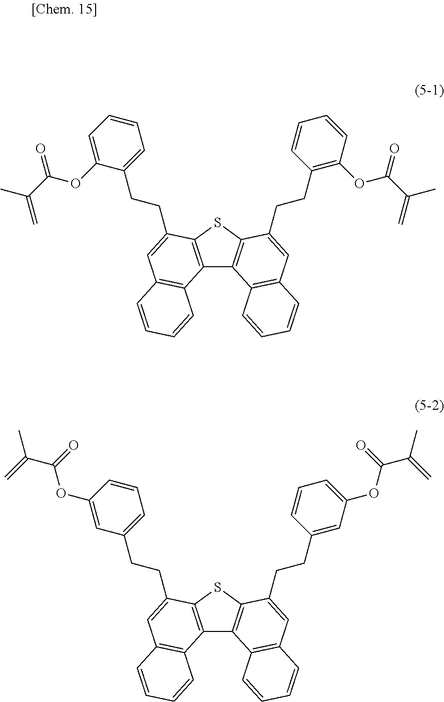

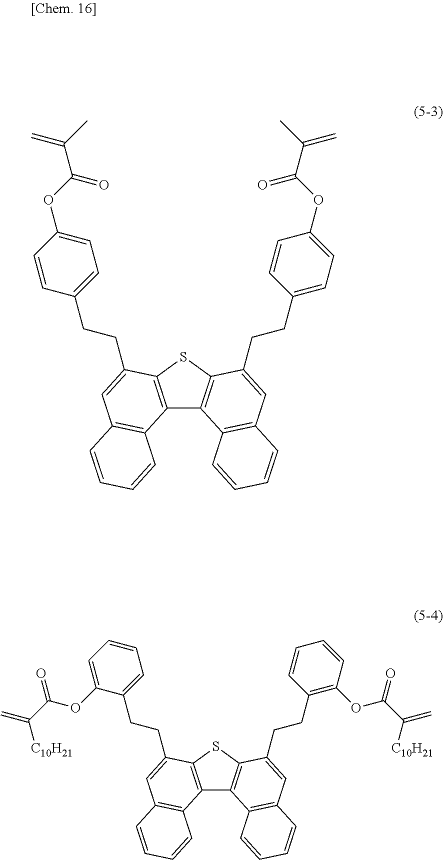

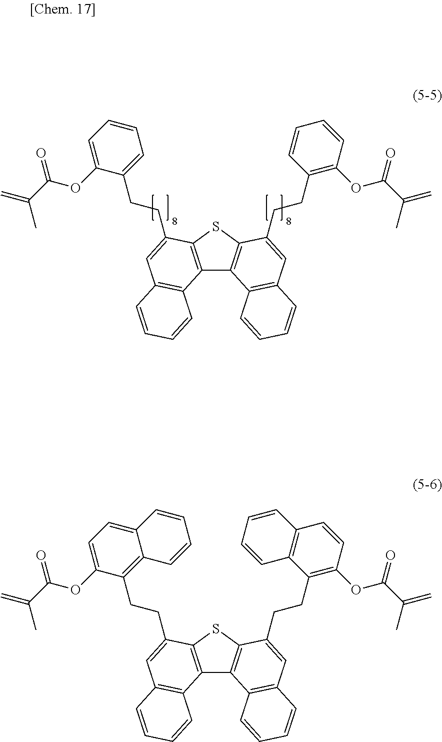

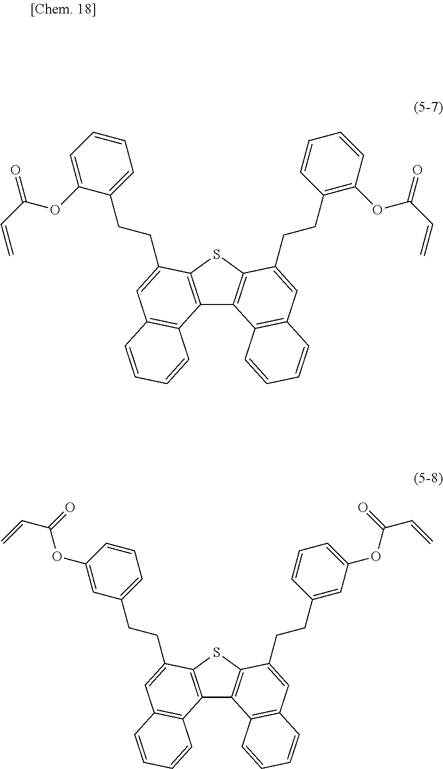

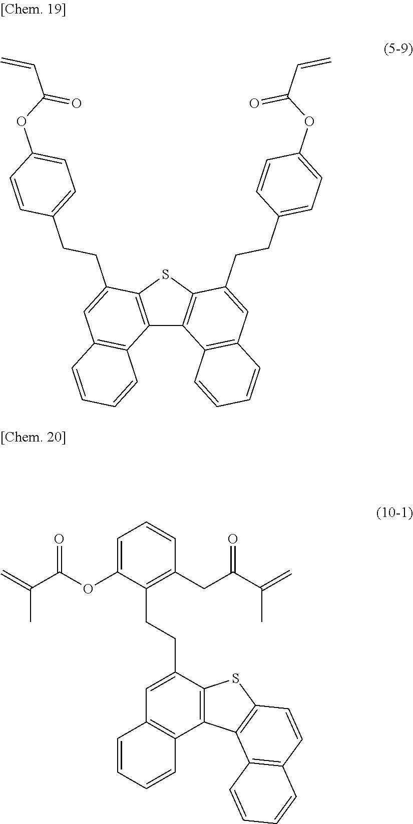

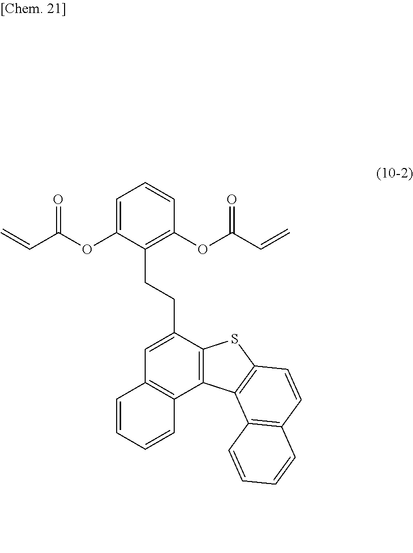

Compounds 300-1 to 300-4 mentioned as preferred examples of the compound represented by the above general formula (1), in which a carbon atom of the carbon skeleton constituting the alkylene group of R²⁰³ in the compound represented by the above general formula (1) is substituted by a hetero atom (oxygen (O), sulfur (S), nitrogen (N), and phosphorus (P)), are set forth below.

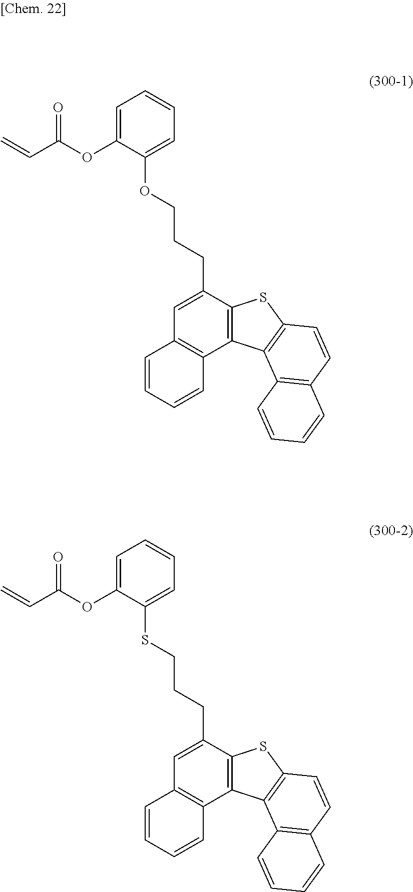

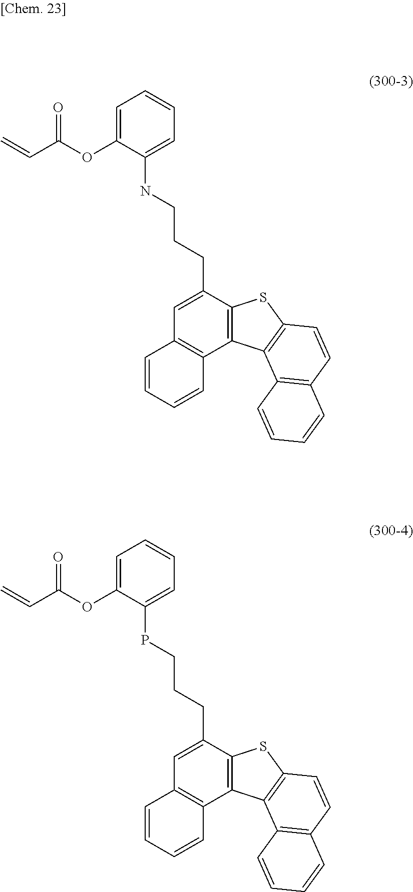

Compounds 300-5 to 300-8 mentioned as preferred examples of the compound represented by the above general formula (1), in which an alkylene group of R²⁰³ in the compound represented by the above general formula (1) is substituted by a halogen atom (fluorine (F), chlorine (Cl), bromine (Br), and iodine (I)), are set forth below.

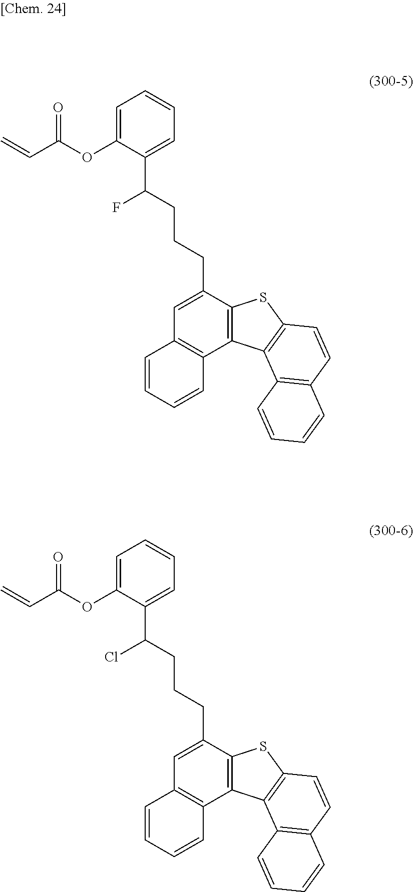

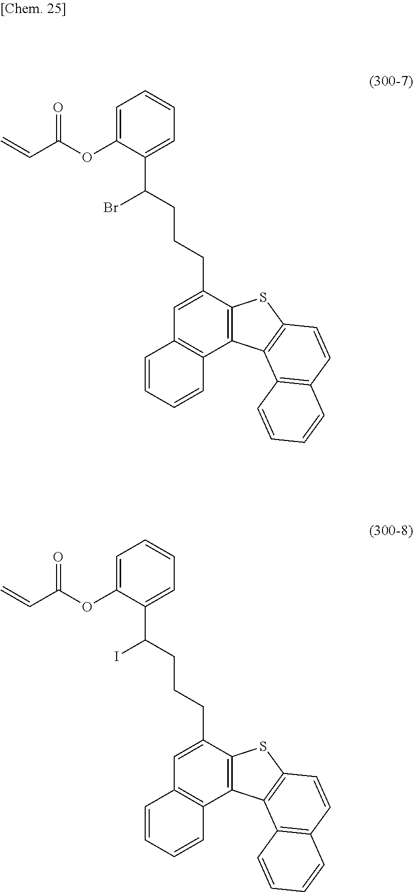

Compounds 400-1 to 400-4 mentioned as preferred examples of the compound represented by the above general formula (1), in which a carbon atom of the carbon skeleton constituting the alkylene group of R²⁰⁴ in the compound represented by the above general formula (1) is substituted by a hetero atom (oxygen (O), sulfur (S), nitrogen (N), and phosphorus (P)), are set forth below.

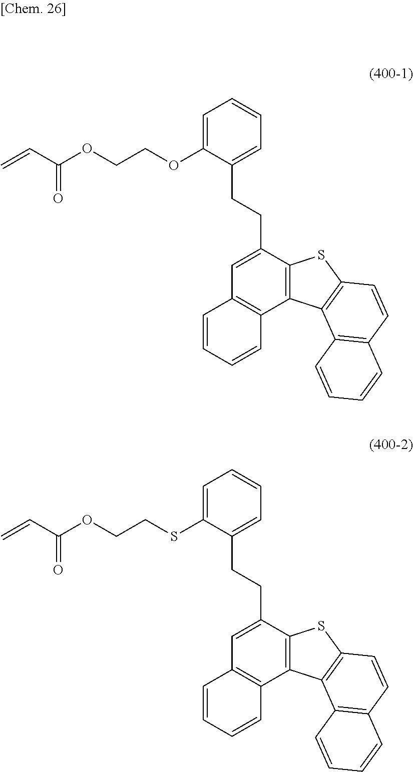

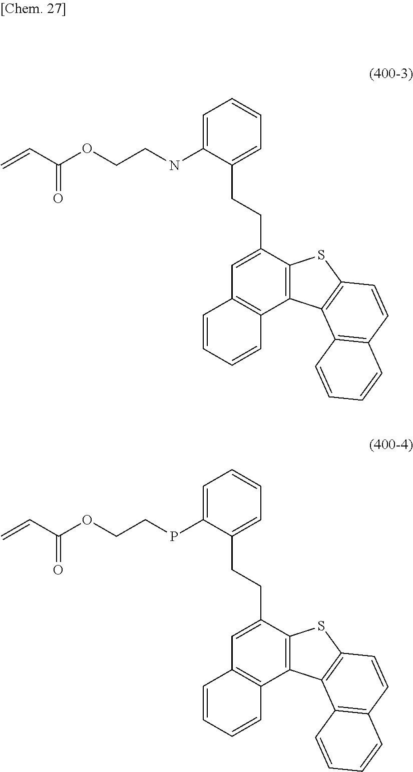

Compounds 400-5 to 400-8 mentioned as preferred examples of the compound represented by the above general formula (1), in which the alkylene group of R²⁰⁴ in the compound represented by the above general formula (1) is substituted by a halogen atom (fluorine (F), chlorine (Cl), bromine (Br), and iodine (I)), are set forth below.

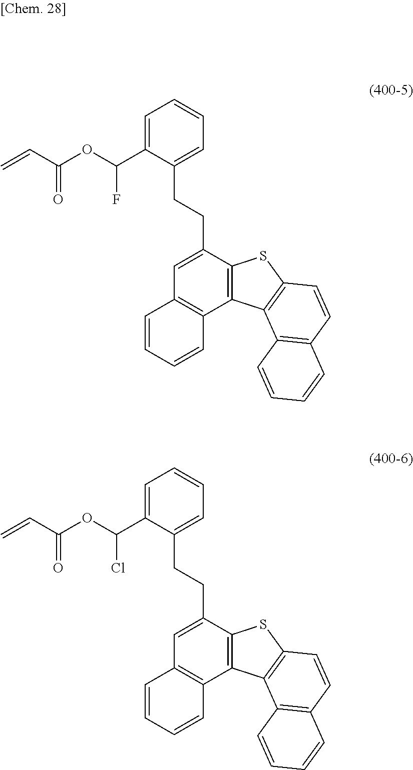

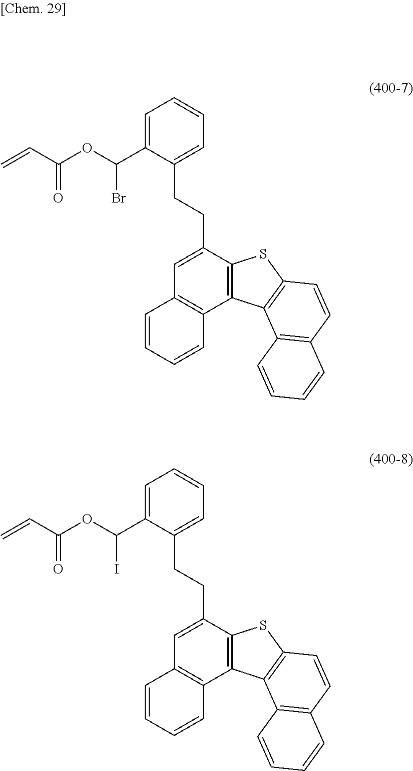

Compounds 500-1 to 500-4 mentioned as preferred examples of the compound represented by the above general formula (1), in which a carbon atom of the carbon skeleton constituting the alkyl group of R²⁰⁵ in the compound represented by the above general formula (1) is substituted by a hetero atom (oxygen (O), sulfur (S), nitrogen (N), and phosphorus (P)), are set forth below.

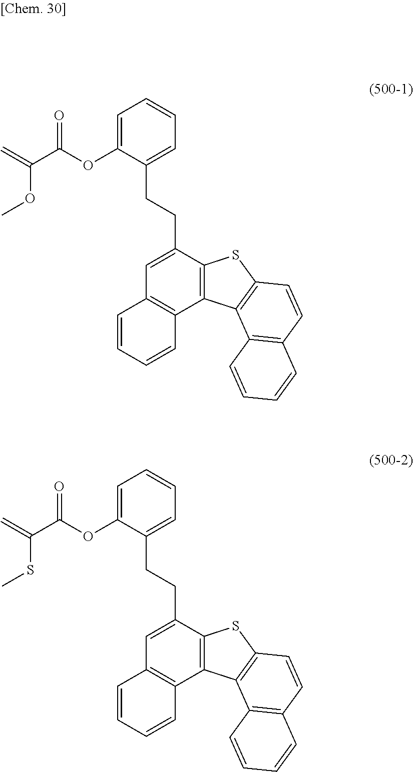

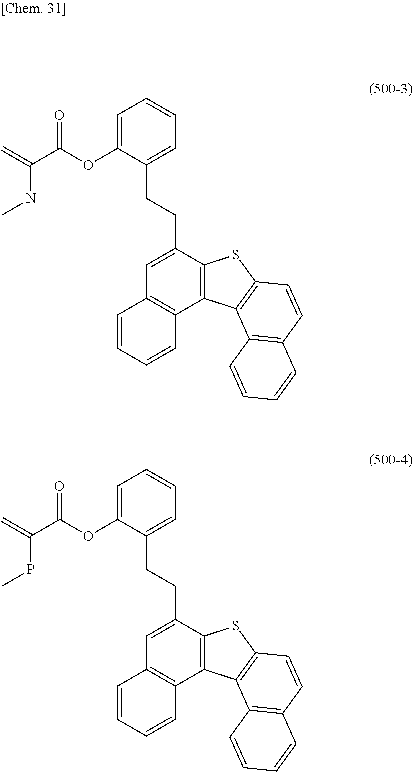

Compounds 500-5 to 500-8 mentioned as preferred examples of the compound represented by the above general formula (1), in which the alkyl group of R²⁰⁵ in the compound represented by the above general formula (1) is substituted by a halogen atom (fluorine (F), chlorine (Cl), bromine (Br), and iodine (I)), are set forth below.

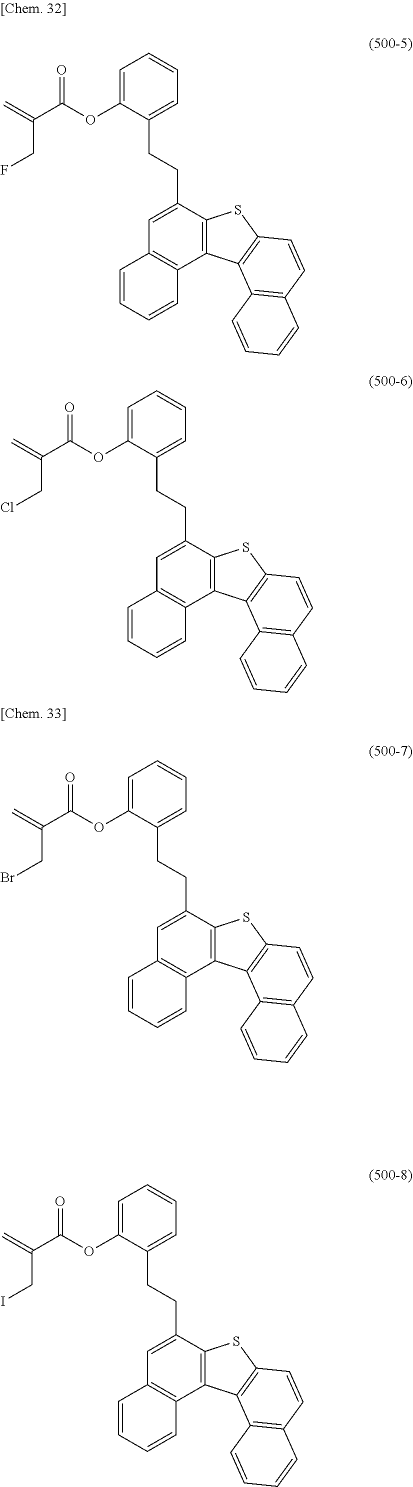

### 3. Second Embodiment (Examples of Polymer)

A polymer of a second embodiment (examples of polymer) according to the present technology is a polymer obtained by polymerizing the compound of the first embodiment of the present technology.

Since the compound of the first embodiment of the present technology is a monofunctional monomer or a multifunctional (bifunctional) monomer, the polymer of the second embodiment according to the present technology can be produced by polymerizing the compound of the first embodiment of the present technology.

The polymer of the second embodiment according to the present technology is able to realize further enhancement of functions of organic materials. In other words, the polymer of the second embodiment according to the present technology simultaneously has high solubility and high transparency and high refractive index and is able to realize further enhancement of functions of organic materials.

### 4. Third Embodiment (Examples of Organic Material)

An organic material of a third embodiment (examples of organic material) according to the present technology is a material that contains the compound of the first embodiment according to the present technology or contains the polymer of the second embodiment of the present technology.

Examples of the organic material of the third embodiment according to the present technology include an organic thin film, an organic lens, a hologram, an organic thin film composition, an organic lens composition, and a hologram recording photosensitive composition. The organic thin film and the organic thin film composition, the organic lens and the organic lens composition, and the hologram and the hologram recording photosensitive composition will be described in detail below.

### [4-1. Organic Thin Film and Organic Thin Film Composition]

The organic thin film composition contains at least the compound of the first embodiment according to the present technology, and the organic thin film can be obtained by subjecting the organic thin film composition to a polymerization treatment such as irradiation with light or heating. In other words, the organic thin film contains the polymer of the second embodiment of the present technology. The organic thin film is what is called a polymer film, and one or more layers of polymer films are normally included in a flat panel display of a liquid crystal display device (hereinafter also referred to as an LCD (Liquid Crystal Display)).

The organic thin film is incorporated in a flat panel display, for example, as a layer constituting a protective film or an antireflection film in an LCD. Besides, the organic thin films are widely used in various fields where surface protection, antireflection, or the like is required, other than in the flat panel display.

The compound of the first embodiment according to the present technology has high solubility and high refractive index and high transparency and is therefore used for an organic thin film (for example, a refractive index gradient film) having a high refractive index surface. In order to obtain an organic thin film (for example, a refractive index gradient film) having a high refractive index surface, the polymer of the compound of the first embodiment having a refractive index of equal to or more than 1.60 is preferably locally present at a surface layer part on a one-side surface side (high refractive index surface side) of the organic thin film (polymer film). The refractive index of the compound of the first embodiment is more preferably equal to or more than 1.65, and further preferably equal to or more than 1.70. On the other hand, the refractive index of the compound of the first embodiment is, for example, equal to or less than 1.80, but may be in excess of 1.80. In addition, as the compound of the first embodiment, two or more different kinds of compounds may be used by mixing in any proportions.

### [4-2. Organic Lens and Organic Lens Composition]

An organic lens composition contains at least the compound of the first embodiment according to the present technology, and an organic lens can be obtained by subjecting the organic lens composition to a polymerization treatment such as irradiation with light or heating. In other words, the organic lens contains the polymer of the second embodiment according to the present technology.

The organic lens has advantages of being lighter in weight, being less liable to crack, and being easier to process than inorganic materials, and the organic lens is used for spectacles and cameras. Since the compound of the first embodiment according to the present technology has high solubility and high refractive index and high transparency, it has advantages of being excellent in convenience in optical uses, such as being formable into a thin lens as compared to glass, in the case of being used as an organic lens.

### [4-3. Hologram and Hologram Recording Photosensitive Composition]

### (Hologram Recording Photosensitive Composition)

A hologram recording photosensitive composition is a composition which at least contains at least two kinds of photopolymerizable monomers, a photopolymerization initiator, a binder resin, and a polymerization inhibitor, and the at least two kinds of photopolymerizable monomers are a monofunctional monomer and a multifunctional monomer. The at least two kinds of photopolymerizable monomers may all be the compounds of the first embodiment according to the present technology, or at least one kind of the at least two kinds of photopolymerizable monomers may be the compound of the first embodiment according to the present technology.

The hologram recording photosensitive composition has high functionality, for example, has a high refractive index modulation amount (Δn), and produces an effect of excellent diffraction characteristics.

In the case where monomers other than the compounds of the first embodiment according to the present technology are used as the at least two kinds of photopolymerizable monomers, any monomers may be used. Examples of a monofunctional or multifunctional monomer include dinaphthothiophene-based monomers in which a group having a polymerizable unsaturated bond is a substituent group on a benzene ring not condensed with the thiophene ring and dinaphthothiophene-based monomers in which a group having a polymerizable unsaturated bond is a substituent group on a benzene ring condensed with the thiophene ring; examples of a multifunctional monomer include triphenylethynylbenzene-based monomers and trinaphthylethynylbenzene-based monomers; and examples of a monofunctional monomer or a multifunctional monomer include carbazole-based monomers and fluorene-based monomers.

The hologram recording photosensitive composition may contain a binder resin. The binder resin is not particularly limited and may be any binder resin; however, the binder resin is preferably a vinyl acetate-based resin, and, particularly, polyvinyl acetate or a hydrolyzate thereof is preferably used. In addition, acrylic resins are preferred, and, particularly, poly(meth)acrylic acid esters or partial hydrolyzates thereof are preferably used.

The hologram recording photosensitive composition may contain a photopolymerization initiator. The photopolymerization initiator is not particularly limited and may be any photopolymerization initiator; however, preferable examples of the photopolymerization initiator include radical polymerization initiators (radical generating agents) or cationic polymerization initiators (acid generating agents) based on any of imidazole, bisimidazole, N-arylglycine, organic azide compound, titanocene, aluminate complex, organic peroxide, N-alkoxypyridinium salt, thioxanthone derivative, sulfonic acid ester, imidosulfonate, dialkyl-4-hydroxysulfonium salt, p-nitrobenzyl arylsulfonate ester, silanol-aluminum complex, (η6-benzene) (η5-cyclopentadienyl)iron(II), ketone, diaryliodonium salt, diaryliodonium organic boron complex, aromatic sulfonium salt, aromatic diazonium salt, aromatic phosphonium salt, triazine compound, and iron arene complex, or polymerization initiators having both of the functions. Note that the photopolymerization initiator contained in the hologram recording photosensitive composition of the first embodiment according to the present technology may be an anionic polymerization initiator (base generating agent).

The hologram recording photosensitive composition may contain a polymerization inhibitor. The polymerization inhibitor is not particularly limited and may be any polymerization inhibitor; however, preferred specific examples of the polymerization inhibitor include quinone-based compounds, hindered phenol-based compounds, benzotriazole-based compounds, and thiazine-based compounds. Examples of the quinone-based compound include hydroquinone, which may be considered as a kind of phenol-based compound. Examples of the thiazine-based compounds include phenothiazine.

The hologram recording photosensitive composition may further contain inorganic fine particles, a plasticizer, a sensitizing dye, a chain transfer agent, and a solvent. Note that the solvent is effective for enhancing film forming property and the like, as well as viscosity adjustment and compatibility control.

### (Method for Preparing Hologram Recording Photosensitive Composition)

The hologram recording photosensitive composition of the first embodiment according to the present technology can be prepared, for example, by adding predetermined amounts of the at least two kinds of photopolymerizable monomers, the photopolymerization initiator, the binder resin, and the polymerization inhibitor to the above-mentioned solvent at room temperature or the like, followed by dissolution and mixing. In addition, the inorganic fine particles, the plasticizer, the sensitizing dye, the chain transfer agent, and the like mentioned above may be added according to the use, purpose, and the like. When the hologram recording photosensitive composition of the first embodiment according to the present technology is formed on a transparent substrate included in a hologram recording medium to be described later, the hologram recording photosensitive composition may be used as a coating liquid.

### (Hologram Recording Medium)

The hologram recording medium is a hologram recording medium which includes at least a photosensitive layer containing the hologram recording photosensitive composition and at least one transparent substrate, in which the photosensitive layer is formed on the at least one transparent substrate. The hologram recording medium may have a three-layer structure in which the photosensitive layer is formed on a first transparent substrate and, further, a second transparent substrate is formed on that main surface of the photosensitive layer on which the first transparent substrate is not formed.

The hologram recording medium has high functionality, for example, has a high refractive index modulation amount (Δn), and produces an effect of excellent diffraction characteristics.

### (Method for Producing Hologram Recording Medium)

The hologram recording medium can be obtained, for example, by applying a coating liquid including a hologram recording photosensitive composition as described above by use of a spin coater, a gravure coater, a comma coater, a bar coater, or the like, followed by drying, to form a photosensitive layer.

### (Hologram)

The hologram is a hologram which has high functionality, for example, has a refractive index modulation amount of equal to or more than 0.06, and has excellent diffraction characteristics and for which the above-mentioned hologram recording medium is used.

### (Method of Producing Hologram)

The hologram can be obtained, for example, by subjecting the hologram recording medium to two-light-speed exposure using a semiconductor laser in a visible light region or the like, followed by irradiating the whole surface of the medium with UV light to cure an uncured photopolymerizable monomer, thereby fixing a refractive index distribution to the hologram recording medium. The conditions of the two-light-speed exposure may be any conditions according to the use and purpose; however, it is desirable to perform interference exposure by setting the light intensity of one flux on the recording medium at 0.1 to 100 mW/cm² and performing exposure for 1 to 1,000 seconds in such a manner that the angle formed between the two fluxes is 0.1° to 179.9°.

### 5. Fourth Embodiment (Example of Image Display Device)

An image display device of a fourth embodiment (example of image display device) according to the present technology is a device including the organic material of the third embodiment of the present technology. The image display device of the fourth embodiment according to the present technology includes the organic material of the third embodiment according to the present technology, and therefore, produces an effect of excellent image display performance.

Examples of the image display device of the fourth embodiment according to the present technology include image display devices such as an eyewear, a holographic screen, a transparent display, a head-mounted display, and a head-up display.

### 6. Fifth Embodiment (Example of Optical Part) and 7. Sixth Embodiment (Example of Optical Device)

An optical part of a fifth embodiment (example of optical part) according to the present technology is a part including the organic material of the third embodiment according to the present technology. The optical part of the fifth embodiment according to the present technology includes the organic material of the third embodiment according to the present technology, and therefore, produces effects of excellent optical characteristics and excellent optical stability.

In addition, an optical device of a sixth embodiment (example of optical device) according to the present technology is an optical device including the organic material of the third embodiment according to the present technology. The optical device of the sixth embodiment according to the present technology includes the organic material of the third embodiment according to the present technology, and therefore, produces effects of excellent optical characteristics and excellent optical stability.

Examples of the optical part of the fifth embodiment according to the present technology and examples of the optical device of the sixth embodiment according to the present technology include an imaging device, an imaging element, a color filter, a diffraction lens, a conductive plate, a spectroscopic element, a hologram sheet, an information recording medium such as an optical disk and a magneto-optical disk, an optical pick-up device, a polarization microscope, and a sensor.

### EXAMPLES

The effects of the present technology will be specifically described below by way of Examples. Note that the scope of the present technology is not to be limited to or by the Examples.

### Example 1

### [Preparation of Compound Represented by Chemical Formula (4-1)]

A compound represented by the following chemical formula (4-1) was synthesized, and the compound represented by the following chemical formula (4-1) was made to be a compound of Example 1.

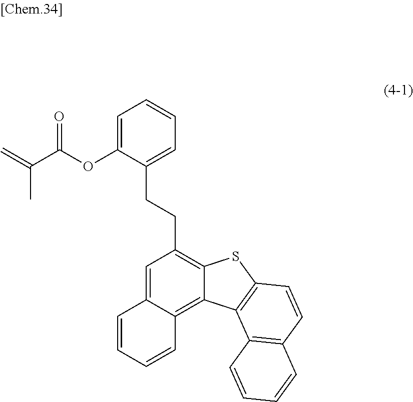 [SMILES: C=C(C)C(=O)Oc1ccccc1CCc1cc2ccccc2c2c1sc1ccc3ccccc3c12]

### [Synthesizing Method for Compound Represented by Chemical Formula (4-1)]

A synthesizing method (synthetic route) for the compound represented by the chemical formula (4-1) is as follows.

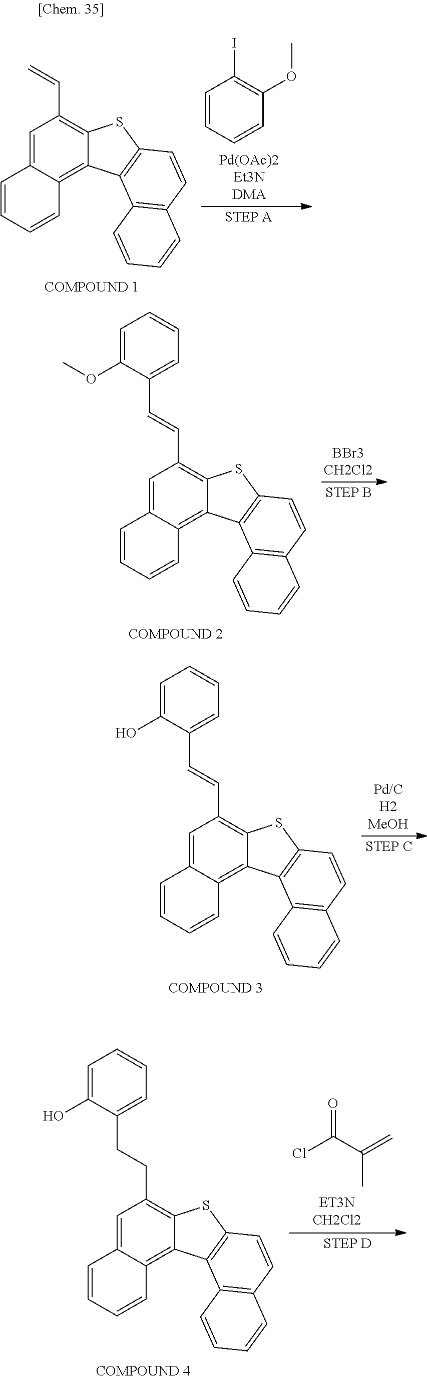

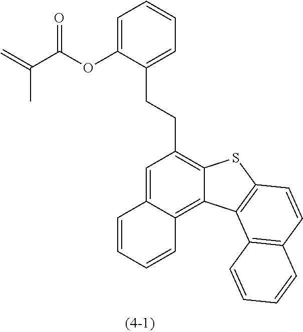

### (Step A)

Step A in the synthetic route depicted above will be described.

In an Ar atmosphere, 6.60 g (21.3 mmol) of Compound 1, 3.25 mL (25.0 mmol) of 2-iodoanisole, and 10.6 g (76.8 mmol) of potassium carbonate were weighed, and then, 170 mL of deoxygenated DMF was added thereto, followed by Ar bubbling for 15 minutes. Next, 6.76 g (21.0 mmol) of TBAB and 0.263 g (1.17 mmol, 0.55 eq.) of Pd(OAc)₂ were added, and the mixture was heated at 110° C. for 4 hours. After being let cool to room temperature, the reaction liquid was poured into 200 mL of iced water. 500 mL of ethyl acetate was added for liquid separation, and the aqueous layer side was extracted with 300 mL of ethyl acetate. After the organic layer was washed with water, the organic layer was dried over magnesium sulfate, and the filtrate was concentrated, dried, and hardened under a reduced pressure. The product was purified by column, to obtain 7.44 g (17.9 mmol) of Compound 2 as a yellow crystal.

### (Step B)

Step B in the synthetic route depicted above will be described.

In an Ar atmosphere, 65.0 mL of super-dehydrated dichloromethane was added to 3.08 g (7.40 mmol) of Compound 2, followed by cooling to 0° C. by ice bath. To the reaction liquid, 16.0 mL (16.0 mmol) of 1.0 M of a dichloromethane solution of BBr₃ was added dropwise, and after stirring the reaction liquid overnight while keeping it on the ice bath, the reaction liquid was poured into 200 mL of iced water, for quenching. After liquid separation, the aqueous layer was extracted with 100 mL of dichloromethane, whereas the organic layer was collectively dried over magnesium sulfate, and the filtrate was concentrated, dried, and hardened under a reduced pressure. The residue was purified by a column, to obtain 915 mg (2.27 mmol) of Compound 3 as a light yellow solid.

### (Step C)

Step C in the synthetic route depicted above will be described.

In an Ar atmosphere, 45.0 mL of ethyl acetate and 13.0 mL of THF were added to 873 mg (2.17 mmol) of Compound 3,150 mg (0.063 mmol) of 10% Pd/C (55% hydrous) was further added, the inside of the apparatus was replaced with hydrogen gas, and the reaction liquid was stirred overnight. After filtering the reaction liquid through celite, the filtrate was concentrated, dried, and hardened under a reduced pressure, to obtain 826 mg (2.04 mmol) of Compound 4 as a light brown solid.

### (Step D)

Step D in the synthetic route depicted above will be described.

In an Ar atmosphere, 15.0 mL of super-dehydrated dichloromethane and 0.370 mL (2.66 mmol) of triethylamine were added to 489 mg (1.21 mmol) of Compound 4, followed by cooling to an internal temperature of 0° C. by ice bath. To the reaction liquid, 0.175 mL (1.85 mmol, 1.53 eq.) of methacryloyl chloride was added dropwise, followed by stirring under ice bath for 1.5 hours, and the reaction liquid was poured into iced water for quenching. After liquid separation, the organic layer was dried over magnesium sulfate, and the filtrate was concentrated, dried, and hardened under a reduced pressure. The residue was purified by a column, to obtain 461 mg (0.43 mmol) of the compound of Example 1 (the compound represented by the chemical formula (4-1)) as an oily light yellow substance.

Then, using NMR, the structure of the compound of Example 1 (the compound represented by the chemical formula (4-1)) was identified. The results of NMR areas follows.

1H NMR (CDCl₃): 2.05 (s, 3H), 3.17 (t, 2H), 3.32 (t, 2H), 5.67 (s, 1H), 6.32 (s, 1H), 7.12 (d, 1H), 7.26 (m, 3H), 7.58 (m, 4H), 7.63 (s, 1H), 7.95 (m, 3H), 8.05 (m, 1H), 8.85 (m, 2H)

### Example 2

### [Preparation of Compound Represented by Chemical Formula (4-2)]

A compound represented by the following chemical formula (4-2) was synthesized, and the compound represented by the following chemical formula (4-2) was made to be a compound of Example 2.

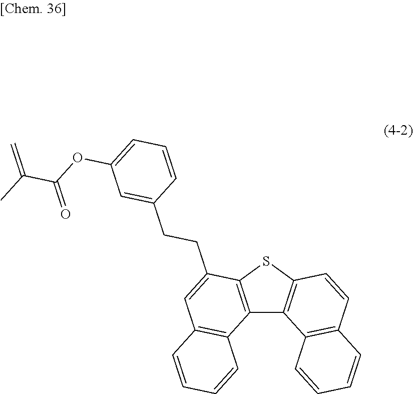 [SMILES: C=C(C)C(=O)Oc1cccc(CCc2cc3ccccc3c3c2sc2ccc4ccccc4c23)c1]

### [Synthesizing Method for Compound Represented by Chemical Formula (4-2)]

A synthesizing method (synthetic route) for the compound represented by the chemical formula (4-2) is similar to the synthesizing method for the compound represented by the chemical formula (4-1), except that 2-iodoanizole used in Step A in the synthesizing method (synthetic route) for the compound represented by the chemical formula (4-1) is replaced by 3-iodoanisole, and the compound represented by the chemical formula (4-2) was synthesized by use of the synthesizing method.

### Example 3

### [Preparation of Compound Represented by Chemical Formula (4-3)]

A compound represented by the following chemical formula (4-3) was synthesized, and the compound represented by the following chemical formula (4-3) was made to be a compound of Example 3.

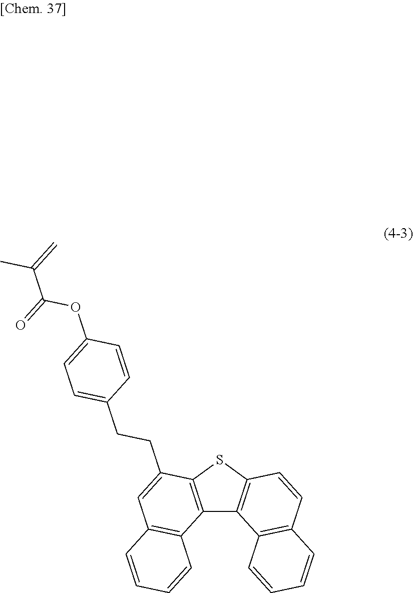 [SMILES: C=C(C)C(=O)Oc1ccc(CCc2cc3ccccc3c3c2sc2ccc4ccccc4c23)cc1]

### [Synthesizing Method for Compound Represented by Chemical Formula (4-3)]

A synthesizing method (synthetic route) for the compound represented by the chemical formula (4-3) is similar to the synthesizing method for the compound represented by the chemical formula (4-1), except that 2-iodoanizole used in Step A in the synthesizing method (synthetic route) for the compound represented by the chemical formula (4-1) is replaced by 4-iodoanisole, and the compound represented by the chemical formula (4-2) was synthesized by use of the synthesizing method.

### Example 4

### [Preparation of Compound Represented by Chemical Formula (4-4)]

A compound represented by the following chemical formula (4-4) was synthesized, and the compound represented by the following chemical formula (4-4) was made to be a compound of Example 4.

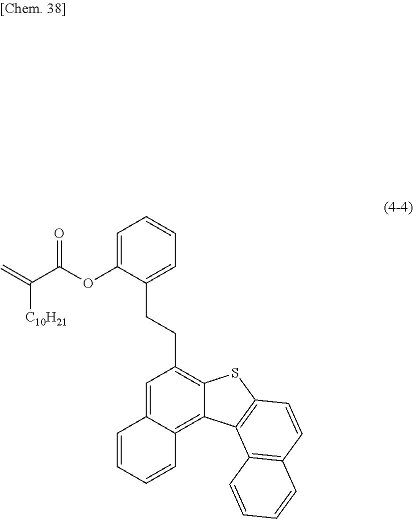

### [Synthesizing Method for Compound Represented by Chemical Formula (4-4)]

A synthesizing method (synthetic route) for the compound represented by the chemical formula (4-4) is similar to the synthesizing method for the compound represented by the chemical formula (4-1), except that methacrylic acid chloride used in Step D in the synthesizing method (synthetic route) for the compound represented by the chemical formula (4-1) is replaced by a compound represented by the following chemical formula (4-4-1), and the compound represented by the chemical formula (4-4) was synthesized by use of the synthesizing method.

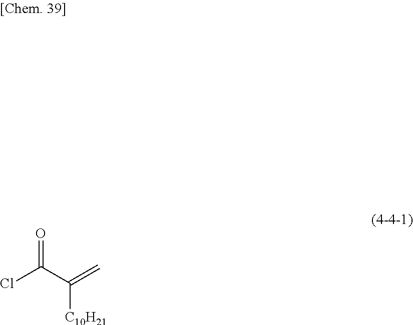

### Example 5

### [Preparation of Compound Represented by Chemical Formula (4-5)]

A compound represented by the following chemical formula (4-5) was synthesized, and the compound represented by the following chemical formula (4-5) was made to be a compound of Example 5.

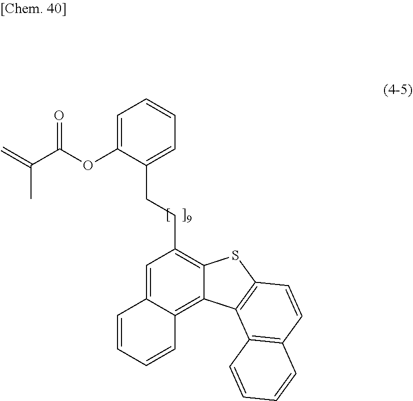

### [Synthesizing Method for Compound Represented by Chemical Formula (4-5)]

A synthesizing method (synthetic route) for the compound represented by the chemical formula (4-5) is similar to the synthesizing method for the compound represented by the chemical formula (4-1), except that 2-iodoanisole used in Step A in the synthesizing method (synthetic route) for the compound represented by the chemical formula (4-1) is replaced by a compound represented by the following chemical formula (4-5-1), and the compound represented by the chemical formula (4-5) was synthesized by use of the synthesizing method.

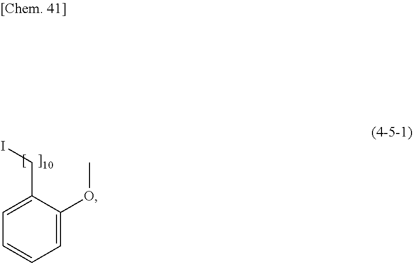

### Example 6

### [Preparation of Compound Represented by Chemical Formula (4-6)]

A compound represented by the following chemical formula (4-6) was synthesized, and the compound represented by the following chemical formula (4-6) was made to be a compound of Example 6.

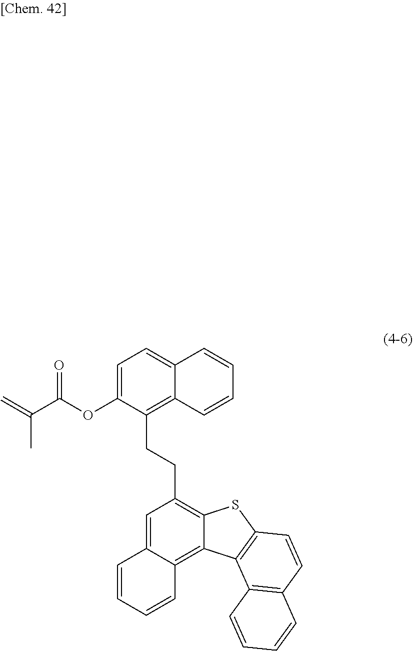 [SMILES: C=C(C)C(=O)Oc1ccc2ccccc2c1CCc1cc2ccccc2c2c1sc1ccc3ccccc3c12]

### [Synthesizing Method for Compound Represented by Chemical Formula (4-6)]

A synthesizing method (synthetic route) for the compound represented by the chemical formula (4-6) is similar to the synthesizing method for the compound represented by the chemical formula (4-1), except that 2-iodoanisole used in Step A in the synthesizing method (synthetic route) for the compound represented by the chemical formula (4-1) is replaced by 1-iodo-2-methoxynaphthalene, and the compound represented by the chemical formula (4-6) was synthesized by use of the synthesizing method.

### Example 13

### [Preparation of Compound Represented by Chemical Formula (10-1)]

A compound represented by the following chemical formula (10-1) was synthesized, and the compound represented by the following chemical formula (10-1) was made to be a compound of Example 13.

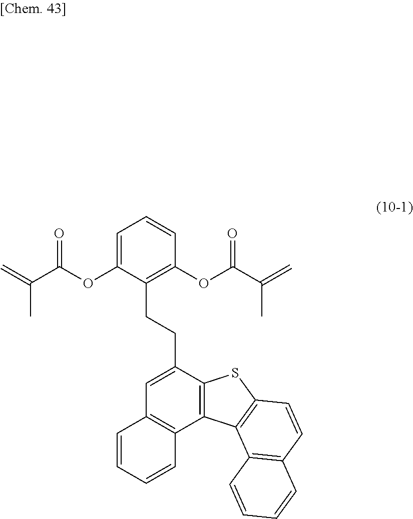 [SMILES: C=C(C)C(=O)Oc1cccc(OC(=O)C(=C)C)c1CCc1cc2ccccc2c2c1sc1ccc3ccccc3c12]

### [Synthesizing Method for Compound Represented by Chemical Formula (10-1)]

A synthesizing method (synthetic route) for the compound represented by the chemical formula (10-1) is as follows.

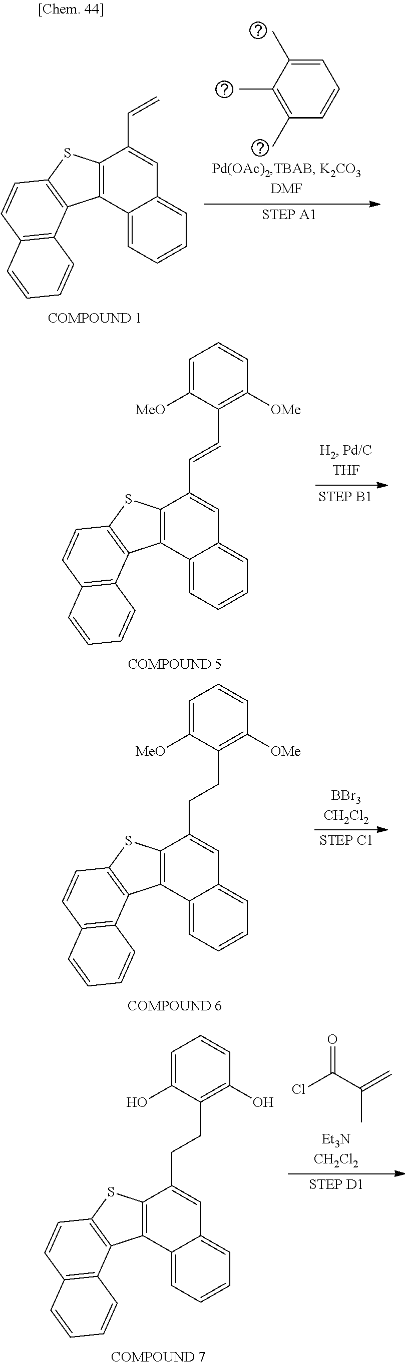

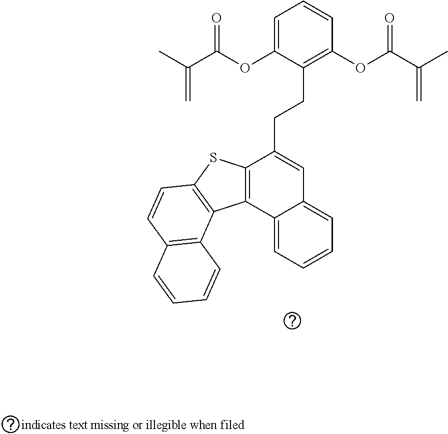 [SMILES: C=C(C)C(=O)Oc1cccc(OC(=O)C(=C)C)c1CCc1cc2ccccc2c2c1sc1ccc3ccccc3c12]

### (Step A1)

Step A1 in the synthetic route depicted above will be described. Step A1 includes the following operations 1 to 14.

1. In an Ar atmosphere, 24. 1 g (77.0 mmol, 1.00 eq.) of Compound 1, 23.7 g (90.0 mmol, 1.16 eq.) of 1-iodo-2,6-dimethoxybenzene, 38.7 g (280 mmol, 3.6 eq.) of potassium carbonate, and 577 g of deoxygenated DMF were added to a test tube.

2. Ar bubbling was conducted for 30 minutes.

3. To the test tube, 24.5 g (76.0 mmol, 0.981 eq.) of TBAB and 982 mg (4.38 mmol, 0.056 eq.) of Pd(OAc)₂ were added.

4. Heating at 110° C. was conducted for 2.5 hours.

5. After being let cool to room temperature, the reaction liquid was poured into 1.2 L of iced water, and 1.0 L of ethyl acetate was added thereto, followed by liquid separation.

6. The organic layer was washed with 1.0 L of water, and the organic layer was filtered.

7. 24.2 g of a black residue was obtained.

8. The black residue was dissolved in 1.0 L of chloroform, and 16.4 g of Si-Thiol was added thereto, followed by stirring at room temperature for 30 minutes.

9. Filtration through celite was conducted.

10. The filtrate was concentrated under a reduced pressure, and heptane was added thereto, followed by slurry filtration.

11. The residue was vacuum dried at 60° C. for 30 minutes.

12. A cream-colored solid in an amount of 21.6 g (48.4 mmol, yield 62.4%, Compound 5) was obtained.

13. The filtrate obtained in operation 10 was concentrated under a reduced pressure, followed by slurry filtration.

14. As a cream-colored solid, Compound 5 in an additional amount of 0.803 g (1.80 mmol, yield 2.3%) was obtained.

### (Step B1)

Step B1 in the synthetic route depicted above will be described. Step B1 includes the following operations 1 to 12.

1. In an Ar atmosphere, a 1-L four-neck flask was charged with 21.1 g (47.2 mmol, 1.00 eq.) of Compound 5 and 420 mL of THF.

2. To the flask, 3.35 g (1.42 mmol, 0.30 eq.) of 10% Pd/C (55% hydrous) was added, and the inside of the apparatus was replaced with hydrogen gas.

3. A hydrogen gas balloon was set, followed by stirring for 6.5 hours in a slightly compressed state.

4. 4.53 g (1.92 mmol, 0.041 eq.) of 10% Pd/C (55% hydrous) was added, and the inside of the apparatus was replaced with hydrogen gas.

5. A hydrogen gas balloon was set, followed by stirring for 1.5 hours in a slightly compressed state.

6. The reaction liquid was filtered through celite.

7. Added to the filtrate was 5.20 g of Si-Thiol, followed by stirring at room temperature for 30 minutes.

8. Further, 5.23 g of Si-Thiol was added, followed by stirring at room temperature for 30 minutes.

9. After filtration, the filtrate was concentrated under a reduced pressure, and heptane was added thereto, followed by slurry filtration.

10. A cream-colored solid in an amount of 12.5 g (27.9 mmol, yield 59.2%, Compound 6) was obtained.

11. The filtrate obtained in operation 9 was concentrated, and heptane was added thereto, followed by slurry filtration.

12. As a cream-colored solid, Compound 6 was obtained in an amount of 5.33 g (11.9 mmol, yield 11.3%).

### (Step C1)

Step C1 in the synthetic route depicted above will be described. Step C1 includes the following operations 1 to 22.

1. In an Ar atmosphere, a 1-L four-neck flask was charged with 16.0 g (35.7 mmol, 1.00 eq.) of Compound 6 and 450 mL of super-dehydrated dichloromethane.

2. Cooling to an internal temperature of 0° C. or below was conducted on an ice bath.

3. To the flask, 150 mL (150 mmol, 4.20 eq.) of a 1.0 M dichloromethane solution of BBr₃ was added dropwise over 40 minutes.

4. Stirring at 5° C. or below was conducted for 1.5 hours.

5. The ice bath was removed and, after warming to room temperature, stirring was conducted for 3.5 hours.

6. The reaction liquid was poured into 1 L of iced water, for quenching.

7. The quenched reaction liquid was filtered, followed by washing with methanol by pouring methanol.

8. A cream-colored solid in an amount of 3.55 g (Compound 6′-1) was obtained.

9. In an Ar atmosphere, a 200-mL four-neck flask was charged with 1.52 g (3.38 mmol, 1.00 eq.) of Compound 6 and 45.0 mL of super-dehydrated dichloromethane.

10. Cooling to an internal temperature of 0° C. or below was conducted on an ice bath.

11. To the flask, 15.0 mL (15.0 mmol, 4.44 eq.) of a 1.0 M dichloromethane solution of BBr₃ was added dropwise over 10 minutes.

12. Stirring at 0° C. or below was conducted for 3 hours.

13. The ice bath was removed and, after warming to room temperature, stirring was conducted for 3 hours.

14. The reaction liquid was poured into 50 mL of water, for quenching.

15. The filtrate obtained in operation 8 and the reaction liquid after the quenching in operation 14 were united together, followed by liquid separation.

16. The aqueous layer was extracted with 200 mL of chloroform.

17. The organic layer was united therewith and the liquid was passed through a phase separator, and the filtrate was concentrated under a reduced pressure.

18. Heptane was added, followed by slurry filtration.

19. The residue obtained was vacuum dried at 60° C. for 30 minutes.

20. A cream-colored solid (Compound 6′-2) was obtained in an amount of 10.7 g.

21. Compound 6′-1 and Compound 6′-2 were united together, followed by vacuum drying at 80° C. for 30 minutes.

22. A cream-colored solid in an amount of 14.0 g (33.4 mmol, Compound 7) was obtained.

### (Step D1)

Step D1 in the synthetic route depicted above will be described. Step D1 includes the following operations 1 to 17.

1. In an Ar atmosphere, a 100-mL eggplant flask was charged with 500 mg (1.20 mmol, 1.00 eq.) of Compound 7, 30.0 mL of super-dehydrated dichloromethane, and 0.750 mL (5.38 mmol, 4.52 eq.) of triethylamine.

2. Cooling to an internal temperature of 0° C. was conducted on an ice bath.

3. To the flask, 0.350 mL (3.70 mmol, 3.11 eq.) of methacryloyl chloride was added dropwise.

4. Stirring on an iced bath was conducted for 2 hours.

5. The reaction liquid was poured into iced water, for quenching.

6. In an Ar atmosphere, a 1-L four-neck flask was charged with 6.50 g (15.5 mmol, 1.00 eq.) of Compound 7, 390 mL of super-dehydrated dichloromethane, and 9.80 mL (70.3 mmol, 4.55 eq.) of triethylamine.

7. Cooling to an internal temperature of 0° C. was conducted on an ice bath,

8. To the flask, 4.60 mL (48.6 mmol, 3.14 eq.) of methacryloyl chloride was added dropwise over 10 minutes.

9. Stirring on an ice bath was conducted for 2.5 hours.

10. The reaction liquid was poured into iced water, for quenching.

11. The reaction liquids after the quenching in operation 5 and operation 10 were united together and, after liquid separation, the aqueous layer was extracted with 100 mL of chloroform.

12. The organic layer was united therewith, followed by washing with water (500 mL×4 times).

13. The organic layer was passed through a phase separator, and the filtrate was concentrated, dried, and hardened under a reduced pressure.

14. The filtrate was concentrated, dried, and hardened under a reduced pressure.

15. The residue was subjected to column purification (column: Biotage SNAP Ultra 340 g×2 (in series), solvent: heptane/ethyl acetate=9/1 (volume ratio)).

16. A fraction containing the target substance singly was recovered and was concentrated, dried, and hardened under a reduced pressure.

17. As awhile solid, a compound represented by the chemical formula (10-1) was obtained in an amount of 5.97 g (10.7 mmol, yield 64.4%).

Then, by use of NMR, the structure of the compound of Example 13 (the compound represented by the chemical formula (10-1)) was identified. The results of NMR are as follows.

1H NMR (CDCl₃): 2.01 (s, 6H), 3.12 (t, 2H), 3.24 (t, 2H), 5.65 (s, 2H), 6.26 (s, 2H), 7.04 (d, 2H), 7.30 (m, 1H), 7.55 (m, 5H), 7.92 (m, 3H), 8.05 (m, 1H), 8.83 (m, 2H)

### Example 14

### [Preparation of Compound Represented by Chemical Formula (10-2)]

A compound represented by the following chemical formula (10-2) was synthesized, and the compound represented by the following chemical formula (10-2) was made to be a compound of Example 14.

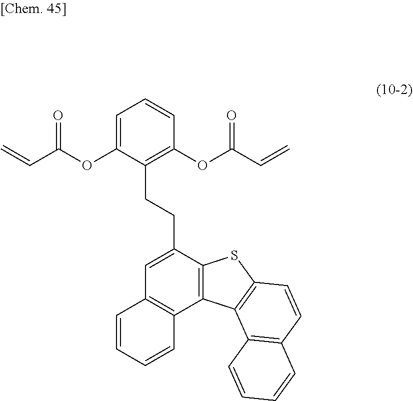 [SMILES: C=CC(=O)Oc1cccc(OC(=O)C=C)c1CCc1cc2ccccc2c2c1sc1ccc3ccccc3c12]

### [Synthesizing Method for Compound Represented by Chemical Formula (10-2)]

A synthesizing method (synthetic route) for the compound represented by the chemical formula (10-2) is as follows.

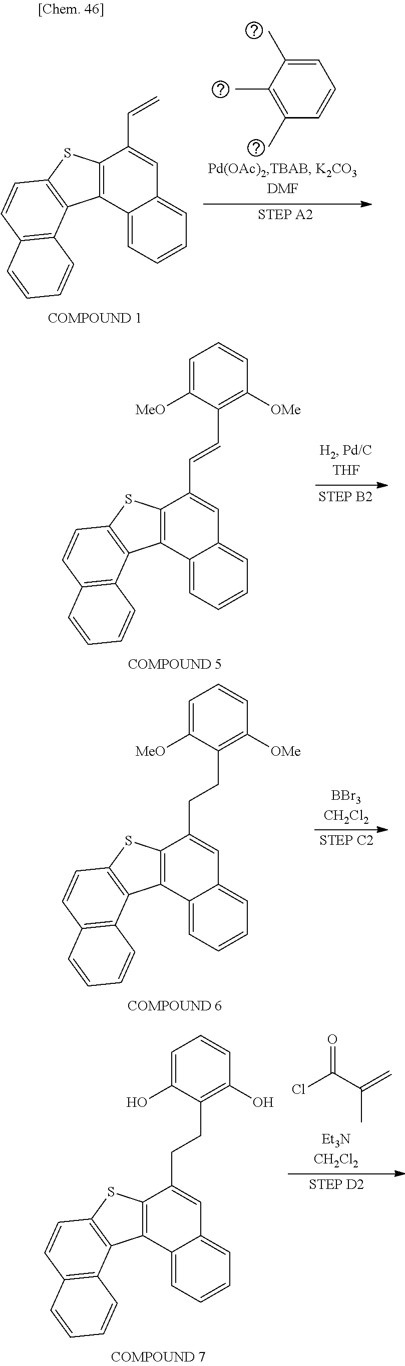

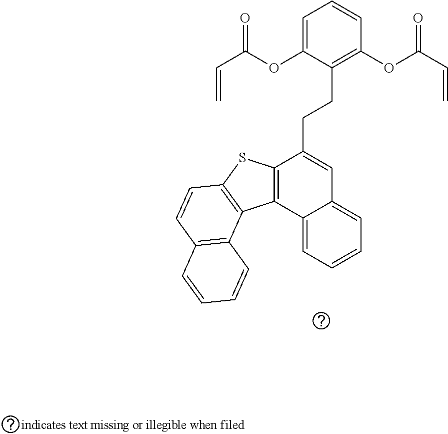 [SMILES: C=CC(=O)Oc1cccc(OC(=O)C=C)c1CCc1cc2ccccc2c2c1sc1ccc3ccccc3c12]

In Step A2 to Step C2, Compound 7 was synthesized by the same method as the synthetic route depicted in regard of the chemical formula (10-1).

### (Step D2)

Step D2 in the synthetic route depicted above will be described. Step D2 includes the following operations 1 to 14.

1. In an Ar atmosphere, a 500-mL four-neck flask was charged with 4.04 g (9.61 mmol, 1.00 eq.) of Compound 7, 240 mL of super-dehydrated chloroform (with amylene added), and 6.00 mL (43.0 mmol, 4.48 eq.) of triethylamine.

2. Cooling to an internal temperature of 0° C. was conducted on an ice bath.

3. To the flask, 2.50 mL (30.8 mmol, 3.20 eq.) of acryloyl chloride was dropped over 10 minutes.

4. Stirring on an ice bath was conducted for 2.5 hours.

5. The reaction liquid was poured into iced water, for quenching.

6. The aqueous layer was extracted with 100 mL of chloroform.

7. The organic layer was united therewith, followed by washing with water (500 mL×three times).

8. The organic layer was passed through a phase separator, and the filtrate was concentrated, dried, and hardened under a reduced pressure.

9. The residue was dissolved in 50 mL of chloroform, and the solution was passed through 31.2 g of silica gel (Kanto Chemical, 60N).

10. Washing with 200 mL of chloroform was conducted.

11. The filtrate was united therewith, and heptane was added thereto, followed by concentration under a reduced pressure.

12. The concentrated residue was subjected to slurry filtration.

13. The filtration residue was vacuum dried at 50° C. for 1 hour.

14. As a light skin colored solid, a compound represented by the chemical formula (10-2) was obtained in an amount of 3.98 g (7.51 mmol, yield 78%).

Then, by use of NMR, the structure of the compound of Example 14 (the compound represented by the chemical formula (10-2)) was identified. The results of NMR were as follows.

1H NMR (CDCl₃): 3.12 (m, 2H), 3.25 (m, 2H), 5.85 (d, 2H), 6.25 (m, 2H), 6.50 (d, 2H), 7.04 (d, 2H), 7.30 (m, 1H), 7.53 (m, 5H), 7.88 (m, 3H), 8.05 (m, 1H), 8.83 (m, 2H)

### Example 15

### [Preparation of Compound Represented by Chemical Formula (11-1)]

A compound represented by the following chemical formula (11-1) was synthesized, and the compound represented by the following chemical formula (11-1) was made to be a compound of Example 15.

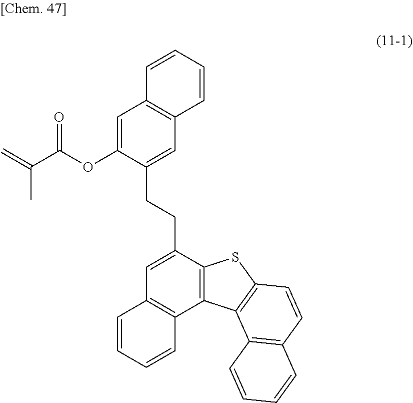 [SMILES: C=C(C)C(=O)Oc1cc2ccccc2cc1CCc1cc2ccccc2c2c1sc1ccc3ccccc3c12]

### [Synthesizing Method for Compound Represented by Chemical Formula (11-1)]

A synthesizing method (synthetic route) for the compound represented by the chemical formula (11-1) is as follows.

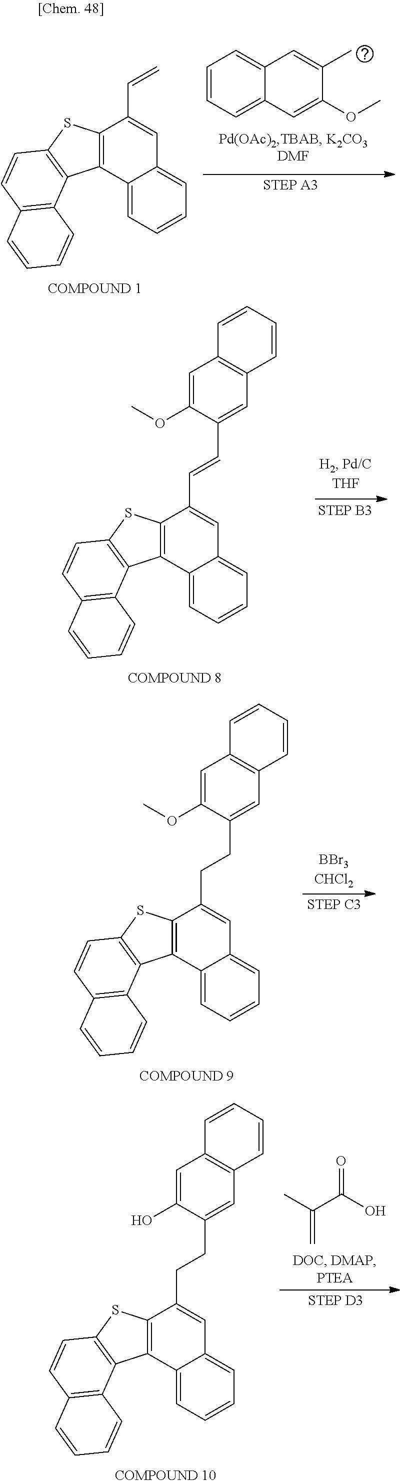

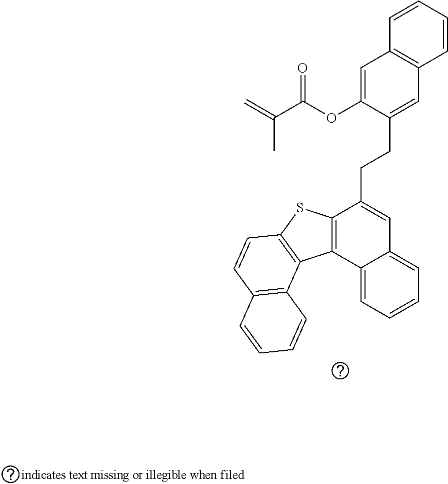 [SMILES: C=C(C)C(=O)Oc1cc2ccccc2cc1CCc1cc2ccccc2c2c1sc1ccc3ccccc3c12]

### (Step A3)

Step A3 in the synthetic route depicted above will be described. Step A3 includes the following operations 1 to 10.

1. In an Ar atmosphere, a 200-mL four-neck flask was charged with 10.9 g (35.2 mmol, 1.00 eq.) of Compound 1, 19.2 g (81.2 mmol, 2.30 eq.) of 2-bromo-3-methoxynaphthalene, 479 g (6557 mmol, 186 eq. eq.) of deoxygenated DMF, and 22.9 g (166 mmol, 4.71 eq.) of potassium carbonate.

2. Ar bubbling was conducted for 28 minutes.

3. To the flask, 12.1 g (37.8 mmol, 1.07 eq.) of TBAB and 1.15 g (5.12 mmol, 0.146 eq.) of Pd(OAc)₂ were added.

4. Stirring under reflux was conducted for 26 hours.

5. After being let cool to room temperature, the reaction liquid was poured into 1 L of water, for quenching.

6. The reaction liquid was suction filtered, followed by washing with 200 mL of ethyl acetate.

7. The crystal was dissolved in 1.2 L of THF, and 19.3 g of Si-Thiol was added thereto, followed by stirring for 35 minutes.

8. The reaction liquid was filtered through celite, followed by washing with 800 mL of THF.

9. After the filtrate was concentrated, 88.8 g of heptane was added thereto.

10. By filtration and drying, Compound 8 as an ocher solid was obtained in an amount of 8.43 g (18.0 mmol, yield 33.0%).

### (Step B3)

Step B3 in the synthetic route depicted above will be described. Step B3 includes the following operations 1 to 7.

1. In an Ar atmosphere, a 2-L four-neck flask was charged with 7.96 g (17.0 mmol, 1.00 eq.) of Compound 8 and 1145 mL (14 mol, 819 eq.) of THF.

2. Stirring was conducted at 30° C. for 1 hour.

3. Added to the flask was 2.26 g (0.956 mmol, 0.0560 eq.) of 10% Pd—C(55% hydrous).

4. The inside of the container was replaced with hydrogen.

5. Filtration with 30.7 g of Si-Thiol and 29.3 g of celite was conducted.

6. The filtrate obtained in operation 5 was concentrated under a reduced pressure, followed by slurry filtration.

7. As a cream-colored solid, Compound 9 was obtained in an amount of 5.22 g (11.1 mmol, yield 65.4%).

### (Step C3)

Step C3 in the synthetic route depicted above will be described. Step C3 includes the following operations 1 to 9.

1. In an Ar atmosphere, a 300-mL four-neck flask was charged with 5.103 g (10.8 mmol, 1.00 eq.) of Compound 9 and 82 mL (1012 mmol, 92.9 eq.) of super-dehydrated chloroform.

2. Cooling to an internal temperature of 5° C. or below was conducted on an ice bath.

3. To the flask, 25.0 mL (25.0 mmol, 2.29 eq.) of a 1.0 M dichloromethane solution of BBr₃ was added dropwise over 5 minutes.

4. The ice bath was removed and, after warming to room temperature, stirring was conducted for 6 hours.

5. The reaction liquid was quenched using 200 mL of iced water, and 100 mL of heptane was added, to obtain 10.9 g of a cream-colored solid.

6. The solid was dissolved by adding 200 mL of THF thereto.

7. Dehydration was conducted over magnesium sulfate, followed by filtration.

8. The filtrate obtained in operation 7 was concentrated under a reduced pressure, and slurry filtration was conducted, to obtain 4.58 g of a cream-colored solid.

9. The solid was dried under a reduced pressure at 40° C. for 40 minutes, to obtain 4.49 g (9.89 mmol, yield 90.8%) of Compound 10 as a cream-colored solid.

### (Step D3)

Step D3 in the synthetic route depicted above will be described. Step D3 includes the following operations 1 to 15.

1. In an Ar atmosphere, a test tube was charged with 3.85 g (8.46 mmol, 1.00 eq.) of Compound 10, 135 mL of deoxygenated toluene, 5.20 g (25.2 mmol, 2.98 eq.) of N,N′-Dicyclohexylcarbodiimide (DCC), 3.11 g (25.5 mmol, 3.01 eq.) of 4-Dimethylaminopyridine (DMAP), and 0.650 g (3.42 mmol, 0.404 eq.) of p-Toluenesulfonic acid (PTSA).

2. To the test tube, 0.895 g (10.4 mmol, 1.23 eq.) of methacrylic acid was added dropwise over 5 minutes.

3. Stirring at room temperature was conducted for 2 hours.

4. Added to the reaction liquid was 100 mL of water, followed by stirring at room temperature for 30 minutes.

5. Filtration was conducted, the filtrate was subjected to liquid separation, and the organic layer was washed twice with 200 mL of water.

6. The organic layer was dried over anhydrous magnesium sulfate and, after filtration, the filtrate was concentrated under a reduced pressure, heptane was added thereto, and slurry filtration was conducted.

7. As a light ocher solid, a compound represented by the chemical formula (11-1) was obtained in an amount of 2.42 g.

8. The filtration residue obtained in operation 5 was suspended in 30 mL of chloroform, followed by filtration.

9. The filtrates obtained in operation 6 and operation 8 were united together, followed by concentration under a reduced pressure, addition of heptane thereto, and slurry filtration.

10. As an ocher solid, the compound represented by the chemical formula (11-1) was obtained in an amount of 1.01 g.

11. The compound represented by the chemical formula (11-1) obtained in operation 7 was dissolved in 110 mL of chloroform, and the solution was passed through 13.1 g of silica gel (Kanto Chemical, 60N), followed by washing with 100 mL of chloroform.

12. The compound represented by the chemical formula (11-1) obtained in operation 10 was dissolved in 40 mL of chloroform, and the solution was passed through 13.1 g of silica gel (Kanto Chemical, 60N), followed by washing with 30 mL of chloroform.

13. The filtrates obtained in operation 11 and operation 12 were united together, and the resultant filtrate was concentrated, dried, and hardened under a reduced pressure.

14. The concentrated residue was dissolved in 30 mL of chloroform, and 30 mL of ethanol was added thereto, followed by concentration under a reduced pressure and slurry filtration.

15. As alight ocher solid, the compound represented by the chemical formula (11-1) was obtained in an amount of 2.81 g (5.38 mmol, yield 63.6%).

Then, by use of NMR, the structure of the compound of Example 15 (the compound represented by the chemical formula (11-1)) was identified. The results of NMR were as follows.

1H NMR (CDCl₃): 2.09 (s, 3H), 3.33 (m, 2H), 3.44 (m, 2H), 5.74 (s, 1H), 6.39 (s, 1H), 7.25 to 8.06 (m, 15H), 8.85 (m, 2H).

### Comparative Example 1

DNTMA (a commercialized product by SUGAI CHEMICAL INDUSTRY CO., LTD.) represented by the following chemical formula (40-1) was made to be a compound of Comparative Example 1.

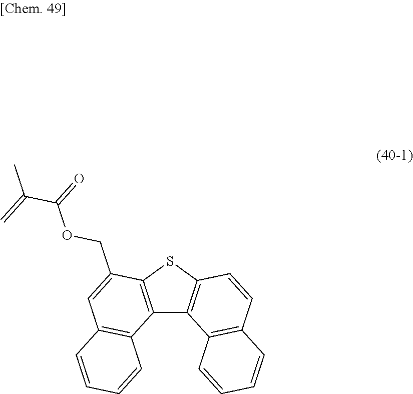 [SMILES: C=C(C)C(=O)OCc1cc2ccccc2c2c1sc1ccc3ccccc3c12]

### Comparative Example 2

EDNTMA (a commercialized product by SUGAI CHEMICAL INDUSTRY CO., LTD.) represented by the following chemical formula (40-2) was made to be a compound of Comparative Example 2.

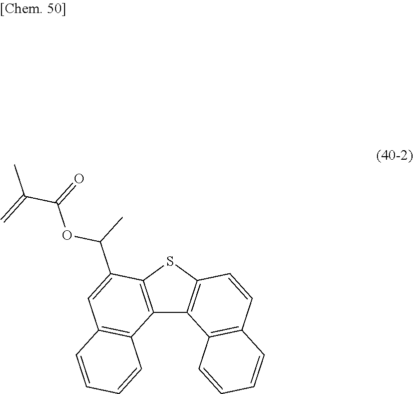 [SMILES: C=C(C)C(=O)OC(C)c1cc2ccccc2c2c1sc1ccc3ccccc3c12]

### Comparative Example 3

6VDNpTh (a commercialized product by SUGAI CHEMICAL INDUSTRY CO., LTD.) represented by the following chemical formula (40-3) was made to be a compound of Comparative Example 3.

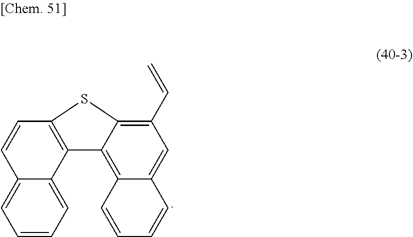 [SMILES: C=Cc1cc2ccccc2c2c1sc1ccc3ccccc3c12]

### <Comparative Example 4]

DNpTh (a commercialized product by SUGAI CHEMICAL INDUSTRY CO., LTD.) represented by the following chemical formula (40-4) was made to be a compound of Comparative Example 4.

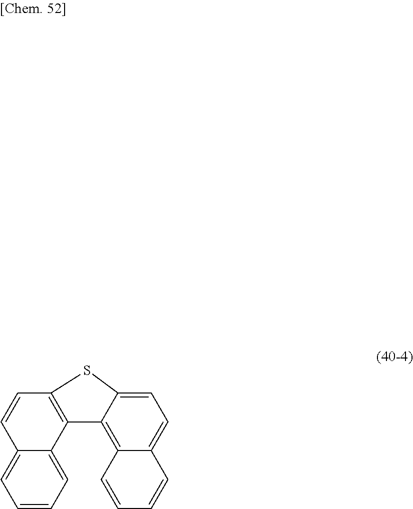 [SMILES: c1ccc2c(c1)ccc1sc3ccc4ccccc4c3c12]

### [Refractive Index Measuring Method and Results]

A refractive index measuring method will be described below.

Acetone solutions of the respective compounds of Examples 1 to 6 and Comparative Examples 1 to 4 were prepared. Average refractive indices of the respective compounds to light of 589 nm at room temperature 25±1° C. were measured by an Abbe's refractometer (ER-1, produced by Erma, Inc.) and were plotted against volume fraction of each compound, to form analytical curves (the densities of the compounds were all 1.00 g/cm³). The analytical curves were extrapolated, and the refractive index when the volume fraction of each compound is 1 was made to be a refractive index of each compound. The results are set forth in Table 1 below.

### [Transparency Measuring Method and Results]

The respective compounds, 10 mg, of Examples 1 to 6 and Comparative Examples 1 to 4 were each mixed with 10 mg of polyvinyl acetate (PVAc, average polymerization degree 5,500), and acetone was added thereto to cause dissolution, thereby preparing resin compositions. Several drops of each of these resin compositions were dropped onto a 2 cm square glass substrate, a film was formed by a spin coater, and acetone as a solvent was evaporated off, to form a resin compatible film (3 μm thick) of each compound. These films were confirmed to be transparent by visual inspection and were subjected to evaluation of transparency. The results are set forth in Table 1 below.

The criterion of transparency evaluation is as follows.

Good . . . Transparency is good.

Poor . . . Coloration is present.

### [Solubility Measuring Method and Results]

The respective compounds, 20 mg, of Examples 1 to 6 and Comparative Examples 1 to 4 were each weighed into a vial, acetone was added thereto, and the whole amount was adjusted to 10 mg, followed by stirring with ultrasonic waves for 30 seconds. When an undissolved portion of the compound was not observed visually, the solubility was assumed to be equal to or more than 20 wt %. When there was an undissolved portion of the compound, a small amount of acetone was added thereto, and then stirring was further conducted for 30 seconds. The above-mentioned work was repeated, and upon dissolution of the whole of the compound, the solubility was calculated from the total amount of the solvent used. The results are set forth in Table 1 below.

TABLE 1

| | | Refractive | | Solubility/ |
|---|---|---|---|---|
| | Compound | index (589 nm) | Transparency | wt % |
| Test | Example 1 | 1.8 | Good | >20 |
| Example 1 | | | | |
| Test | Example 2 | 1.8 | Good | >20 |
| Example 2 | | | | |
| Test | Example 3 | 1.8 | Good | >20 |
| Example 3 | | | | |
| Test | Example 4 | 1.7 | Good | >20 |
| Example 4 | | | | |
| Test | Example 5 | 1.7 | Good | >20 |
| Example 5 | | | | |
| Test | Example 6 | 1.81 | Good | >20 |
| Example 6 | | | | |
| Test | Comparative | 1.73 | Good | <10 |
| Example 7 | Example 1 | | | |
| Test | Comparative | 1.71 | Poor | <11 |
| Example 8 | Example 2 | | | |
| Test | Comparative | 1.8 | Poor | <1.5 |
| Example 9 | Example 3 | | | |
| Test | Comparative | 1.81 | Good | <0.1 |
| Example 10 | Example 4 | | | |
| Test | Example 13 | 1.73 | Good | >20 |
| Example 11 | | | | |
| Test | Example 14 | 1.72 | Good | >20 |
| Example 12 | | | | |
| Test | Example 15 | 1.81 | Good | >20 |
| Example 13 | | | | |

For example, in the case of using the compound as a monomer material for hologram, it is desirable that the solubility of the compound is more than 20 wt %. Therefore, the compounds of Test Examples 1 to 6 (the compounds of Examples 1 to 6) and Test Examples 11 to 13 (the compounds of Examples 13 to 15) are preferably used as a monomer material for hologram.

The evaluation of transparency of Test Examples 1 to 6 (Examples 1 to 6) and Test Examples 11 to 13 (Examples 13 to 15) was that transparency is good (evaluated as Good). On the other hand, the evaluation of transparency of Test Example 8 (the compound of Comparative Example 2) and Test Example 9 (the compound of Comparative Example 3) was that coloration is present (evaluated as Poor), and the resin compatible films formed in Test Example 8 and Test Example 9 were both colored in a light yellow color.

### [Production of Hologram Recording Photosensitive Composition and Hologram, and Evaluation of Hologram]

Using Compounds 4-1 to 4-6 prepared in Examples 1 to 6, Compounds 10-1 to 10-2 and 11-1 prepared in Examples 13 to 15, and Compounds 40-1 to 40-3 of Comparative Examples 1 to 3, hologram recording photosensitive compositions and holograms were produced, and evaluation of the holograms produced was conducted.

First, an evaluating technique for diffraction characteristics will be described.

### <Evaluating Technique for Diffraction Characteristics>

### (Calculating Method for Refractive Index Modulation Amount)

A refractive index modulation amount (hereinafter also referred to as Δn) was calculated based on the Kogelnik's theoretical formula.

The Kogelnik's theoretical formula refers to the following formula described in Bell Syst. Tech. J., 48, 2909 (1969).

Kogelnik's theoretical formula:

η=tan h²(π(Δn)d/λ cos θ)

Here, η is a diffraction efficiency, d is a film thickness of a photosensitive layer (photopolymer), λ is a recording laser wavelength, and θ is an incidence angle of recording laser light into a photosensitive material.

### Example 7

(Preparation of Hologram Recording Photosensitive Composition 7) 0.3 g of bisphenoxyethanolfluorene dimethacrylate (“EA-0200,” produced by Osaka Gas Chemicals Co., Ltd.) as a multifunctional (difunctional) photopolymerizable monomer, 1.4 g of Compound 4-1 (the compound of Example 1) as a monofunctional photopolymerizable monomer, 0.5 g of polyvinyl acetate (“SN-55T,” produced by Denki Kagaku Kogyo Kabushiki Kaisha) as a binder resin, 0.09 g of 4-isopropyl-4′-methyldiphenyliodonium-tetrakis(pentafluorophenyl) borate (“D1,” produced by Tokyo Chemical Industry Co., Ltd.) as a photopolymerization initiator, 0.003 g of hydroquinone (“HQ,” produced by FUJIFILM Wako Pure Chemical Corporation) as a polymerization inhibitor, 1 g of diethyl sebacate (“SDE,” produced by FUJIFILM Wako Pure Chemical Corporation) as a plasticizer, 0.08 g of rose bengal (“RB,” produced by SIGMA ALDRICH) as a sensitizing dye, 0.02 g of 2-mercaptobenzooxazole (“2-MBO,” produced by Tokyo Chemical Industry Co., Ltd.) as a chain transfer agent, and 8 g of acetone as a solvent were mixed with one another at normal temperature, to prepare Hologram Recording Photosensitive Composition 7.

### (Fabrication of Hologram Recording Medium 7)

Hologram Recording Photosensitive Composition 7 was applied onto a 2.5 μm-thick polyvinyl alcohol film by a bar coater in such a manner that a dried film thickness of 3 μm would be obtained, and then a thin film surface of Photosensitive Layer 7 including Hologram Recording Photosensitive Composition Resin 7 was press bonded onto a 1.0 mm-thick glass substrate, to fabricate Hologram Recording Medium 7.

### (Fabrication of Hologram 7)

Hologram Recording Medium 7 was subjected to two-light-speed exposure using a semiconductor laser of an exposure wavelength of 532 nm, and the whole surface of Hologram Recording Medium 7 was irradiated with UV light to cure an uncured monomer, thereby fixing a refractive index distribution to Medium 7. The conditions of the two-light-speed exposure were interference exposure for 30 seconds by setting the light intensity of one light flux on the recording medium at 2.6 mW/cm² in such a manner that the angle formed between the two light fluxes was 7°. By this, Hologram Recording Medium 7 was formed with the refractive index distribution, thereby fabricating Hologram 7.

### (Evaluation of Hologram 7)

The refractive index modulation amount (Δn) of thus fabricated Hologram 7 was calculated by use of the Kogelnik's theoretical formula. The refractive index modulation amount (Δn) was 0.09.

### Example 8

### (Preparation of Hologram Recording Photosensitive Composition 8)

In Example 8, Hologram Recording Photosensitive Composition 8 was prepared by the same method as in Example 7 by using the same materials in the same amounts as in Example 7, except that 1.4 g of Compound 4-2 (the compound of Example 2) was used as a monofunctional photopolymerizable monomer.

### (Fabrication of Hologram Recording Medium 8)

Using Hologram Recording Photosensitive Composition 8 thus prepared, Hologram Recording Medium 8 was fabricated by the same method as in Example 7.

### (Fabrication of Hologram 8)

Using Hologram Recording Medium 8 thus fabricated, Hologram 8 was fabricated by the same method as in Example 7.

### (Evaluation of Hologram 8)

The refractive index modulation amount (Δn) of Hologram 8 thus fabricated was determined by the same method as in Example 7. An of Hologram 8 was 0.092.

### Example 9

### (Preparation of Hologram Recording Photosensitive Composition 9)

In Example 9, Hologram Recording Photosensitive Composition 9 was prepared by the same method as in Example 7 by using the same materials in the same amounts as in Example 7, except that 1.4 g of Compound 4-3 (the compound of Example 3) was used as a monofunctional photopolymerizable monomer.

### (Fabrication of Hologram Recording Medium 9)

Using Hologram Recording Photosensitive Composition 9 thus prepared, Hologram Recording Medium 9 was fabricated by the same method as in Example 7.

### (Fabrication of Hologram 9)

Using Hologram Recording Medium 9 thus fabricated, Hologram 9 was fabricated by the same method as in Example 7.

### (Evaluation of Hologram 9)

The refractive index modulation amount (Δn) of Hologram 9 thus fabricated was determined by the same method as in Example 7. An of Hologram 9 was 0.091.

### Example 10

### (Preparation of Hologram Recording Photosensitive Composition 10)

In Example 10, Hologram Recording Photosensitive Composition 10 was prepared by the same method as in Example 7 by using the same materials in the same amounts as in Example 7, except that 1.4 g of Compound 4-4 (the compound of Example 4) was used as a monofunctional photopolymerizable monomer.

### (Fabrication of Hologram Recording Medium 10)

Using Hologram Recording Photosensitive Composition 10 thus prepared, Hologram Recording Medium 10 was fabricated by the same method as in Example 7.

### (Fabrication of Hologram 10)

Using Hologram Recording Medium 10 thus fabricated, Hologram 10 was fabricated by the same method as in Example 7.

### (Evaluation of Hologram 10)

The refractive index modulation amount (Δn) of Hologram 10 thus fabricated was determined by the same method as in Example 7. An of Hologram 10 was 0.068.

### Example 11

### (Preparation of Hologram Recording Photosensitive Composition 11)

In Example 11, Hologram Recording Photosensitive Composition 11 was prepared by the same method as in Example 7 by using the same materials in the same amounts as in Example 7, except that 1.4 g of Compound 4-5 (the compound of Example 5) was used as a monofunctional photopolymerizable monomer.

### (Fabrication of Hologram Recording Medium 11)

Using Hologram Recording Photosensitive Composition 11 thus prepared, Hologram Recording Medium 11 was fabricated by the same method as in Example 7.

### (Fabrication of Hologram 11)

Using Hologram Recording Medium 11 thus fabricated, Hologram 11 was fabricated by the same method as in Example 7.

### (Evaluation of Hologram 11)

The refractive index modulation amount (Δn) of Hologram 11 thus fabricated was determined by the same method as in Example 7. An of Hologram 11 was 0.068.

### Example 12

### (Preparation of Hologram Recording Photosensitive Composition 12)

In Example 12, Hologram Recording Photosensitive Composition 12 was prepared by the same method as in Example 7 by using the same materials in the same amounts as in Example 7, except that 1.4 g of Compound 4-6 (the compound of Example 6) was used as a monofunctional photopolymerizable monomer.

### (Fabrication of Hologram Recording Medium 12)

Using Hologram Recording Photosensitive Composition 12 thus prepared, Hologram Recording Medium 12 was fabricated by the same method as in Example 7.

### (Fabrication of Hologram 12)

Using Hologram Recording Medium 12 thus fabricated, Hologram 12 was fabricated by the same method as in Example 7.

### (Evaluation of Hologram 12)

The refractive index modulation amount (Δn) of Hologram 12 thus fabricated was determined by the same method as in Example 7. An of Hologram 12 was 0.092.

### Example 16

### (Preparation of Hologram Recording Photosensitive Composition 16)

In Example 16, Hologram Recording Photosensitive Composition 16 was prepared by the same method as in Example 7 by using the same materials in the same amounts as in Example 7, except that 1.4 g of Compound 10-1 (the compound of Example 13) was used as a monofunctional photopolymerizable monomer.

### (Fabrication of Hologram Recording Medium 16)

Using Hologram Recording Photosensitive Composition 13 thus prepared, Hologram Recording Medium 13 was fabricated by the same method as in Example 7.

### (Fabrication of Hologram 16)

Using Hologram Recording Medium 16 thus fabricated, Hologram 16 was fabricated by the same method as in Example 7.

### (Evaluation of Hologram 16)

The refractive index modulation amount (Δn) of Hologram 13 thus fabricated was determined by the same method as in Example 7. An of Hologram 13 was 0.072.

### Example 17

### (Preparation of Hologram Recording Photosensitive Composition 17)

In Example 17, Hologram Recording Photosensitive Composition 17 was prepared by the same method as in Example 7 by using the same materials in the same amounts as in Example 7, except that 1.4 g of Compound 10-2 (the compound of Example 14) was used as a monofunctional photopolymerizable monomer.

### (Fabrication of Hologram Recording Medium 17)

Using Hologram Recording Photosensitive Composition 17 thus prepared, Hologram Recording Medium 17 was fabricated by the same method as in Example 7.

### (Fabrication of Hologram 17)

Using Hologram Recording Medium 17 thus fabricated, Hologram 17 was fabricated by the same method as in Example 7.

### (Evaluation of Hologram 17)

The refractive index modulation amount (Δn) of Hologram 17 thus fabricated was determined by the same method as in Example 7. An of Hologram 14 was 0.074.

### Example 18

### (Preparation of Hologram Recording Photosensitive Composition 18)

In Example 18, Hologram Recording Photosensitive Composition 18 was prepared by the same method as in Example 7 by using the same materials in the same amounts as in Example 7, except that 1.4 g of Compound 11-1 (the compound of Example 15) was used as a monofunctional photopolymerizable monomer.

### (Fabrication of Hologram Recording Medium 18)

Using Hologram Recording Photosensitive Composition 18 thus prepared, Hologram Recording Medium 18 was fabricated by the same method as in Example 7.

### (Fabrication of Hologram 18)

Using Hologram Recording Medium 18 thus fabricated, Hologram 18 was fabricated by the same method as in Example 7.

### (Evaluation of Hologram 18)

The refractive index modulation amount (Δn) of Hologram 18 thus fabricated was determined by the same method as in Example 7. An of Hologram 15 was 0.092.

### Comparative Example 5

### (Preparation of Hologram Recording Photosensitive Composition 50)

In Comparative Example 5, Hologram Recording Photosensitive Composition 50 was prepared by the same method as in Example 7 by using the same materials in the same amounts as in Example 7, except that 0.88 g of Compound 40-1 (the compound of Comparative Example 1) was used as a monofunctional photopolymerizable monomer. The amount in which Compound 40-1 was used (0.88 g) was smaller than the amount in which Compounds 4-1 to 4-6 used in Examples 7 to 12, Compounds 10-1 to 10-2 used in Examples 16 to 17, and Compound 11-1 used in Example 18 were used (1.4 g), because the amount in which Compound 40-1 is dissolved in a solvent is smaller than the amounts in which Compounds 4-1 to 4-6, Compounds 10-1 to 10-2, and Compound 11-1 are dissolved in the solvent, that is, the solubility of Compound 40-1 is lower than the that of each of Compounds 4-1 to 4-6, 10-1 to 10-2, and 11-1 (see Table 1). Note that the amount (0.88 g) in which Compound 40-1 was used is a limit value (saturation amount) of dissolution of the compound in a solvent.

### (Fabrication of Hologram Recording Medium 50)

Using Hologram Recording Photosensitive Composition 50 thus prepared, Hologram Recording Medium 50 was fabricated by the same method as in Example 7.

### (Fabrication of Hologram 50)

Using Hologram Recording Medium 50 thus fabricated, Hologram 50 was fabricated by the same method as in Example 7.

### (Evaluation of Hologram 50)

The refractive index modulation amount (Δn) of Hologram 50 thus fabricated was determined by the same method as in Example 7. An of Hologram 50 was 0.055.

### Comparative Example 6

### (Preparation of Hologram Recording Photosensitive Composition 60)

In Comparative Example 6, Hologram Recording Photosensitive Composition 60 was prepared by the same method as in Example 7 by using the same materials in the same amounts as in Example 7, except that 1 g of Compound 40-2 (the compound of Comparative Example 2) was used as a monofunctional photopolymerizable monomer. The amount in which Compound 40-2 was used (1 g) was smaller than the amount in which Compounds 4-1 to 4-6 used in Examples 7 to 12, Compounds 10-1 to 10-2 used in Examples 16 to 17, and Compound 11-1 used in Example 18 were used (1.4 g), because the amount in which Compound 40-2 is dissolved in a solvent is smaller than the amounts in which Compounds 4-1 to 4-6, Compounds 10-1 to 10-2, and Compound 11-1 are dissolved in the solvent, that is, the solubility of Compound 40-2 is lower than the that of each of Compounds 4-1 to 4-6, 10-1 to 10-2, and 11-1 (see Table 1). Note that the amount (1 g) in which Compound 40-2 was used is a limit value (saturation amount) of dissolution of the compound in a solvent.

### (Fabrication of Hologram Recording Medium 60)

Using Hologram Recording Photosensitive Composition 60 thus prepared, Hologram Recording Medium 60 was fabricated by the same method as in Example 7.

### (Fabrication of Hologram 60)

Using Hologram Recording Medium 60 thus fabricated, Hologram 60 was fabricated by the same method as in Example 7.

### (Evaluation of Hologram 60)

The refractive index modulation amount (Δn) of Hologram 60 thus fabricated was determined by the same method as in Example 7. An of Hologram 60 was 0.055.

### Comparative Example 7

### (Preparation of Hologram Recording Photosensitive Composition 70)

In Comparative Example 7, Hologram Recording Photosensitive Composition 70 was prepared by the same method as in Example 7 by using the same materials in the same amounts as in Example 7, except that 0.1 g of Compound 40-3 (the compound of Comparative Example 3) was used as a monofunctional photopolymerizable monomer. The amount in which Compound 40-3 was used (0.1 g) was smaller than the amount in which Compounds 4-1 to 4-6 used in Examples 7 to 12, Compounds 10-1 to 10-2 used in Examples 16 to 17, and Compound 11-1 used in Example 18 were used (1.4 g), because the amount in which Compound 40-3 is dissolved in a solvent is smaller than the amounts in which Compounds 4-1 to 4-6, Compounds 10-1 to 10-2, and Compound 11-1 are dissolved in the solvent, that is, the solubility of Compound 40-3 is lower than the that of each of Compounds 4-1 to 4-6, 10-1 to 10-2, and 11-1 (see Table 1). Note that the amount (0.1 g) in which Compound 40-3 was used is a limit value (saturation amount) of dissolution of the compound in a solvent.

### (Fabrication of Hologram Recording Medium 70)

Using Hologram Recording Photosensitive Composition 70 thus prepared, Hologram Recording Medium 70 was fabricated by the same method as in Example 7.

### (Fabrication of Hologram 70)

Using Hologram Recording Medium 70 thus fabricated, Hologram 70 was fabricated by the same method as in Example 7.

### (Evaluation of Hologram 70)

The refractive index modulation amount (Δn) of Hologram 70 thus fabricated was determined by the same method as in Example 7. An of Hologram 70 was 0.025.

The compositions (the materials and amounts used), hologram exposure conditions, and the results of diffraction characteristics (refractive index modulation amount (Δn)) in regard of Examples 7 to 12 are set forth together in Table 2 below.

The compositions (the materials and amounts used), hologram exposure conditions, and the results of diffraction characteristics (refractive index modulation amount (Δn)) in regard of Comparative Examples 5 to 7 are set forth together in Table 3 below.

TABLE 2

| | | Example | Example | Example | Example | Example | Example | Example | Example | Example |
|---|---|---|---|---|---|---|---|---|---|---|
| | | 7 | 8 | 9 | 10 | 11 | 12 | 16 | 17 | 18 |
| Composition | Photopo1ymerizable | | | | | | | | | |
| | monomer | | | | | | | | | |
| | Compound 4-1 | 1.4 | | | | | | | | |
| | Compound 4-2 | | 1.4 | | | | | | | |
| | Compound 4-3 | | | 1.4 | | | | | | |
| | Compound 4-4 | | | | 1.4 | | | | | |
| | Compound 4-5 | | | | | 1.4 | | | | |
| | Compound 4-6 | | | | | | 1.4 | | | |
| | Compound 10-1 | | | | | | | 1.4 | | |
| | Compound 10-2 | | | | | | | | 1.4 | |
| | Compound 11-1 | | | | | | | | | 1.4 |
| | EA-0200 | 0.3 | 0.3 | 0.3 | 0.3 | 0.3 | 0.3 | 0.3 | 0.3 | 0.3 |
| | Binder resin | | | | | | | | | |
| | SN-55T | 0.5 | 0.5 | 0.5 | 0.5 | 0.5 | 0.5 | 0.5 | 0.5 | 0.5 |
| | Photopolymerization | | | | | | | | | |
| | initiator | | | | | | | | | |
| | DI | 0.09 | 0.09 | 0.09 | 0.09 | 0.09 | 0.09 | 0.09 | 0.09 | 0.09 |
| | Polymerization | | | | | | | | | |
| | inhibitor | | | | | | | | | |
| | HQ | 0.003 | 0.003 | 0.003 | 0.003 | 0.003 | 0.003 | 0.003 | 0.003 | 0.003 |
| | Plasticizer | | | | | | | | | |
| | SDE | 1 | 1 | 1 | 1 | 1 | 1 | 1 | 1 | 1 |
| | Sensitizing dye | | | | | | | | | |
| | RB | 0.08 | 0.08 | 0.08 | 0.08 | 0.08 | 0.08 | 0.08 | 0.08 | 0.08 |
| | Chain transfer agent | | | | | | | | | |
| | 2-MBO | 0.02 | 0.02 | 0.02 | 0.02 | 0.02 | 0.02 | 0.02 | 0.02 | 0.02 |
| | Solvent | | | | | | | | | |
| | Acetone | 8 | 8 | 8 | 8 | 8 | 8 | 8 | 8 | 8 |
| Exposure | Exposure wavelength/nm | 532 | 532 | 532 | 532 | 532 | 532 | 532 | 532 | 532 |
| conditions | One surface exposure | 2.6 | 2.6 | 2.6 | 2.6 | 2.6 | 2.6 | 2.6 | 2.6 | 2.6 |
| | intensity/mW · cm−2 | | | | | | | | | |
| | Exposure time/sec | 30 | 30 | 30 | 30 | 30 | 30 | 30 | 30 | 30 |
| Diffraction | Δn | 0.090 | 0.092 | 0.091 | 0.068 | 0.068 | 0.092 | 0.074 | 0.072 | 0.092 |
| characteristic | | | | | | | | | | |

TABLE 3

| | | Compar- | Compar- | Compar- |
|---|---|---|---|---|
| | | ative | ative | ative |
| | | Example 7 | Example 8 | Example 9 |
| Composition | Photopolymerizable | | | |
| | monomer | | | |
| | Compound 40-1 | 0.88 | | |
| | Compound 40-2 | | 1 | |
| | Compound 40-3 | | | 0.1 |
| | EA-0200 | 0.3 | 0.3 | 0.3 |
| | Binder resin | | | |
| | SN-55T | 0.5 | 0.5 | 0.5 |
| | Photopolymerization | | | |
| | initiator | | | |
| | DI | 0.09 | 0.09 | 0.09 |
| | Polymerization | | | |
| | inhibitor | | | |
| | HQ | 0.003 | 0.003 | 0.003 |
| | Plasticizer | | | |
| | SDE | 1 | 1 | 1 |
| | Sensitizing dye | | | |
| | RB | 0.08 | 0.08 | 0.08 |
| | Chain transfer | | | |
| | agent | | | |
| | 2-MBO | 0.02 | 0.02 | 0.02 |
| | Solvent | | | |
| | Acetone | 8 | 8 | 8 |
| Exposure | Exposure | 532 | 532 | 532 |
| conditions | wavelength/nm) | | | |
| | One surface | 2.6 | 2.6 | 2.6 |
| | exposure | | | |
| | intensity/mW · cm−2 | | | |
| | Exposure time/sec | 30 | 30 | 30 |
| Diffraction | Δn | 0.055 | 0.055 | 0.025 |
| characteristic | | | | |

Note that the present technology is not to be limited to or by the above-described Embodiments and Examples, and various modifications are possible within such ranges as not to depart from the gist of the present technology.

In addition, the effects described herein are merely illustrative and not limitative, and other effects may be present.

Besides, the present technology may also take the following configurations.

[1]

A compound represented by the following general formula (1).

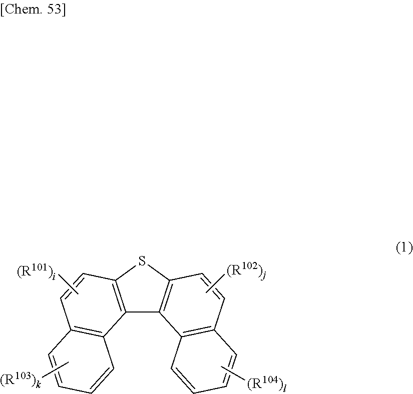

(In the general formula (1), R¹⁰¹ to R¹⁰⁴ are each independently a univalent substituent group represented by the following general formula (2), and i to 1 are each independently an integer of 0 or 1, provided that i to 1 are not simultaneously 0.)

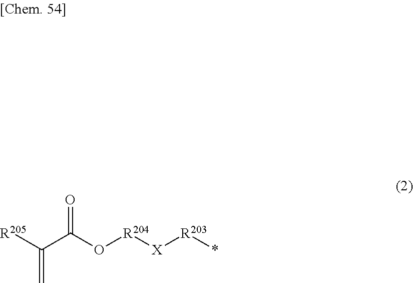

(In the general formula (2), R²⁰³ and R²⁰⁴ are each independently a single bond or a straight chain or branched substituted or unsubstituted alkylene group represented by CₙH₂ₙ (n is an integer of equal to or greater than 1), R²⁰⁵ is hydrogen or a straight chain or branched substituted or unsubstituted alkyl group represented by CₙH₂ₙ₊₁ (n is an integer of equal to or greater than 1), and X is a bivalent aromatic group. The bivalent aromatic group is unsubstituted or has at least one substituent group. Two parts for bonding to R²⁰³ and R²⁰⁴, possessed by the bivalent aromatic group, may be on any bondable carbon in the aromatic group. Represented by * in R¹⁰¹ to R¹⁰² is a part for bonding with carbon that is bondable in a benzene ring condensed with a thiophene ring in the general formula (1). Represented by * in R¹⁰³ to R¹⁰⁴ is a part for bonding with carbon that is bondable in the benzene ring not condensed with the thiophene ring in the general formula (1).)

[2]

The compound according to [1], in which R²⁰³ is a single bond or a straight chain or branched substituted or unsubstituted alkylene group represented by CₙH₂ₙ (n is an integer of 1≤n≤10).

[3]

The compound according to [1], in which R²⁰³ is a single bond or a straight chain or branched substituted or unsubstituted alkylene group represented by CₙH₂ₙ (n is an integer of 1≤n≤3).

[4]

The compound according to any one of [1] to [3], in which R²⁰⁴ is a single bond or a straight chain or branched substituted or unsubstituted alkylene group represented by CₙH₂ₙ (n is an integer of 1≤n≤10).

[5]

The compound according to any one of [1] to [4], in which R²⁰⁵ is hydrogen or a straight chain or branched substituted or unsubstituted alkyl group represented by CₙH₂ₙ₊₁ (n is an integer of 1≤n≤10).

[6]

The compound according to any one of [1] to [5], in which X is a bivalent aromatic group represented by the following chemical formulas (3-1) to (3-8).

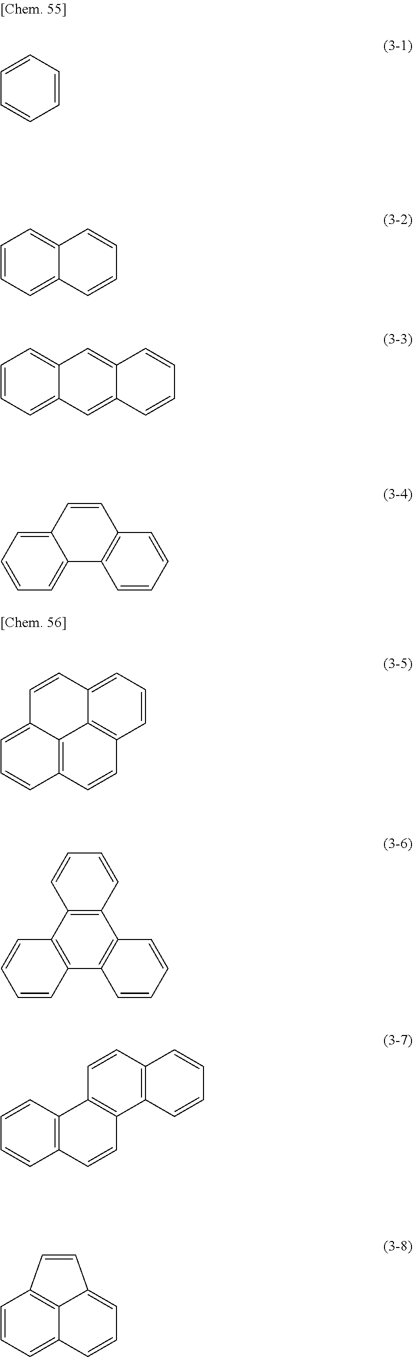

The compound according to any one of [1] to [5], in which the bivalent aromatic group is a monocyclic arylene group, and two parts for bonding to R²⁰³ and R²⁰⁴, possessed by the monocyclic arylene group, are in a relation of ortho positions, meta positions, or para positions.

[8]

The compound according to any one of [1] to [7], in which at least one of R¹⁰¹ or R¹⁰² is adjacent to a carbon atom adjacent to a sulfur atom in the general formula (1) and is bonded to bondable carbon in the benzene ring condensed with the thiophene ring in the general formula (1).

[9]

An organic material including the compound according to any one of [1] to [8].

[10]

The organic material according to [9], that is an organic thin film, an organic lens, or a hologram.

[11]

The organic material according to [9], that is an organic thin film composition, an organic lens composition, or a hologram recording photosensitive composition.

[12]

A polymer obtained by polymerizing the compound according to any one of [1] to [8].

[13]

An organic material including the polymer according to [12].

[14]

The organic material according to [13], that is an organic thin film, an organic lens, or a hologram.

[15]

The organic material according to [13], that is an organic thin film composition, an organic lens composition, or a hologram recording photosensitive composition.

Further, the present technology may also take the following configurations.

[16]

A compound represented by the following general formula (1).

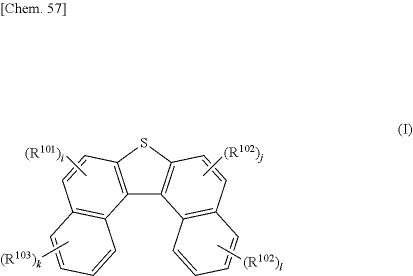

(In the general formula (1), R¹⁰¹ to R¹⁰⁴ are each independently a univalent substituent group represented by the following general formula (2-1), and i to 1 are each independently an integer of 0 or 1, provided that i to 1 are not simultaneously 0.)

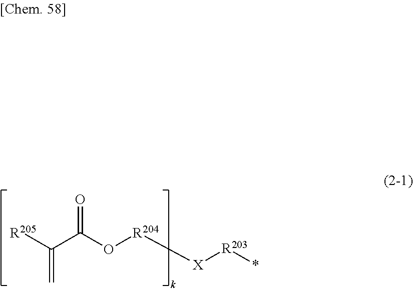

(In the general formula (2-1), R²⁰³ and R²⁰⁴ are each independently a single bond or a straight chain or branched substituted or unsubstituted alkylene group represented by CₙH₂ₙ (n is an integer of equal to or greater than 1), and R²⁰⁵ is hydrogen or a straight chain or branched substituted or unsubstituted alkyl group represented by CₙH₂ₙ₊₁ (n is an integer of equal to or greater than 1). Represented by k is an integer of equal to or greater than 1, and X is a bivalent or more-valent aromatic group. If carbon not bonded to R²⁰³ and R²⁰⁴ is present in the bivalent or more-valent aromatic group, the carbon is unsubstituted or has at least one substituent group. In addition, a part for bonding to R²⁰³ and at least one part for bonding to R²⁰⁴, possessed by the bivalent or more-valent aromatic group, may be any bondable carbon in the aromatic group. Represented by * in R¹⁰¹ to R¹⁰² is a part for bonding with carbon that is bondable in a benzene ring condensed with a thiophene ring in the general formula (1). Represented by * in R¹⁰³ to R¹⁰⁴ is a part for bonding with carbon that is bondable in the benzene ring not condensed with the thiophene ring in the general formula (1).)

[17]

The compound according to [16], in which at least one carbon atom of at least one carbon skeleton, of carbon skeletons constituting the alkylene groups of R²⁰³ and R²⁰⁴ and the alkyl group of R²⁰⁵, is substituted by a hetero atom.

[18]

The compound according to [16] or [17], in which at least one hydrogen atom, of hydrogen atoms constituting the alkylene group of R²⁰³, hydrogen atoms constituting the alkylene group of R²⁰⁴, and hydrogen atoms constituting the alkyl group of R²⁰⁵, is substituted by a halogen atom.

[19]

The compound according to any one of [16] to [18], in which R²⁰³ is a single bond or a straight chain or branched substituted or unsubstituted alkylene group represented by CₙH₂ₙ (n is an integer of 1≤n≤10).

[20]

The compound according to [19], in which at least one carbon atom of a carbon skeleton constituting the alkylene group of R²⁰³ is substituted by a hetero atom.

[21]

The compound according to [19] or [20], in which at least one hydrogen atom of the hydrogen atoms constituting the alkylene group of R²⁰³ is substituted by a halogen atom.

[22]

The compound according to any one of [16] to [18], in which R²⁰³ is a single bond or a straight chain or branched substituted or unsubstituted alkylene group represented by CₙH₂ₙ (n is an integer of 1≤n≤3).

[23]

The compound according to [22], in which at least one carbon atom of a carbon skeleton constituting the alkylene groups of R²⁰³ is substituted by a hetero atom.

[24]

The compound according to [22] or [23], in which at least one hydrogen atom of the hydrogen atoms constituting the alkylene group of R²⁰³ is substituted by a halogen atom.

[25]

The compound according to any one of [16] to [18], in which R²⁰⁴ is a single bond or a straight chain or branched substituted or unsubstituted alkylene group represented by CₙH₂ₙ (n is an integer of 1≤n≤10).

[26]

The compound according to [25], in which at least one carbon atom of a carbon skeleton constituting the alkylene group of R²⁰⁴ is substituted by a hetero atom.

[27]

The compound according to [25] or [26], in which at least one hydrogen atom of the hydrogen atoms constituting the alkylene group of R²⁰⁴ is substituted by a halogen atom.

[28]

The compound according to any one of [16] to [18], in which R²⁰⁵ is hydrogen or a straight chain or branched substituted or unsubstituted alkyl group represented by CₙH₂ₙ 1 (n is an integer of 1≤n≤10).

[29]

The compound according to [28], in which at least one carbon atom of a carbon skeleton constituting the alkyl group of R²⁰⁵ is substituted by a hetero atom.

[30]

The compound according to [28] or [29], in which at least one hydrogen atom of the hydrogen atoms constituting the alkyl group of R²⁰⁵ is substituted by a halogen atom.

[31]

The compound according to any one of [16] to [30], in which X is a bivalent or more-valent aromatic group represented by the following chemical formulas (3-1) to (3-8).

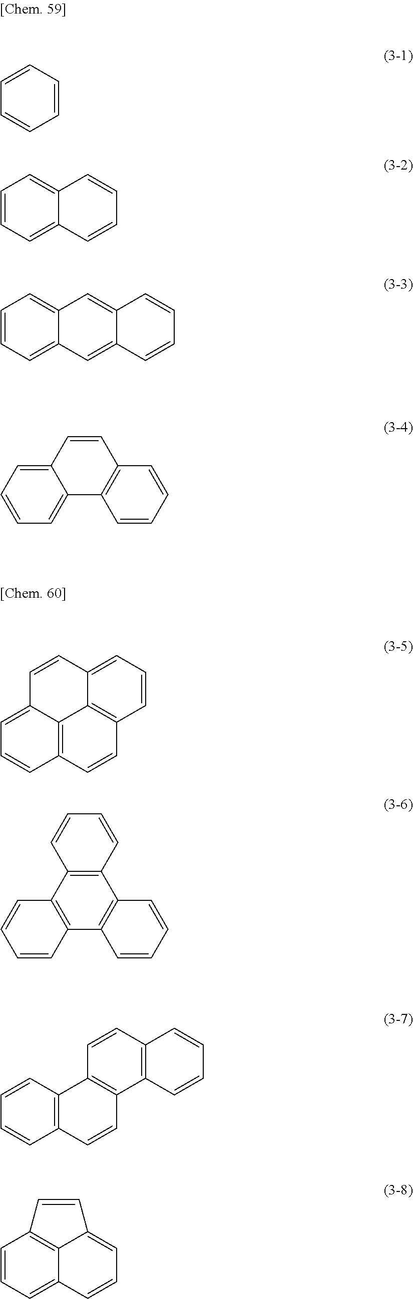

The compound according to any one of [16] to [31], in which k is an integer of 1, and X is a bivalent aromatic group.

[33]

The compound according to [32], in which the bivalent aromatic group is a monocyclic arylene group, and two parts for bonding to R²⁰³ and R²⁰⁴, possessed by the monocyclic arylene group, are in a relation of ortho positions, meta positions, or para positions.

[34]

The compound according to [32], in which the bivalent aromatic group is a polycyclic arylene group, and two parts for bonding to R²⁰³ and R²⁰⁴, possessed by the polycyclic arylene group, are any two bondable carbon atoms in the polycyclic arylene group.

[35]

The compound according to any one of [16] to [31], in which k is 2, and X is a trivalent aromatic group.

[36]

The compound according to [35], in which the trivalent aromatic group is a monocyclic trivalent aromatic group, and a part for bonding to R²⁰³ and one part of two parts for bonding to the two R²⁰⁴ groups, possessed by the monocyclic trivalent aromatic group, are in a relation of ortho positions, meta positions, or para positions.

[37]

The compound according to [35] or [36], in which the trivalent aromatic group is a monocyclic trivalent aromatic group, and two parts for bonding to R²⁰⁴, possessed by the monocyclic trivalent aromatic group, are in a relation of ortho positions, meta positions, or para positions.

[38]

The compound according to [35], in which the trivalent aromatic group is a polycyclic trivalent aromatic group, and a part for bonding to R²⁰³ and one part of two parts for bonding to the two R²⁰⁴ groups, possessed by the polycyclic trivalent aromatic group, are any two bondable carbon atoms in the polycyclic trivalent aromatic group.

[39]

The compound according to [35] or [38], in which the trivalent aromatic group is a polycyclic trivalent aromatic group, and two parts for bonding to R²⁰⁴, possessed by the polycyclic trivalent aromatic group, are any two bondable carbon atoms in the polycyclic trivalent aromatic group.

[40]

The compound according to any one of [16] to [39], in which at least one of R¹⁰¹ or R¹⁰² is adjacent to a carbon atom adjacent to a sulfur atom in the general formula (1) and is bonded to bondable carbon in the benzene ring condensed with the thiophene ring in the general formula (1).

[41]

An organic material including the compound according to any one of [16] to [40].

[42]

The organic material according to [41], that is an organic thin film, an organic lens, or a hologram.

[43]

The organic material according to [41], that is an organic thin film composition, an organic lens composition, or a hologram recording photosensitive composition.

[44]

A polymer obtained by polymerizing the compound according to any one of [16] to [40].

[45]

An organic material including the polymer according to [44].

[46]

The organic material according to [45], that is an organic thin film, an organic lens, or a hologram.

[47]

The organic material according to [45], that is an organic thin film composition, an organic lens composition, or a hologram recording photosensitive composition.

[48]

An image display device including the organic material according to [41].

[49]

An optical part including the organic material according to [41].

[50]

An optical device including the organic material according to [41].

[51]

An image display device including the organic material according to [45].

[52]

An optical part including the organic material according to [45].

[53]

An optical device including the organic material according to [45].

1. A compound represented by the following general formula (1). 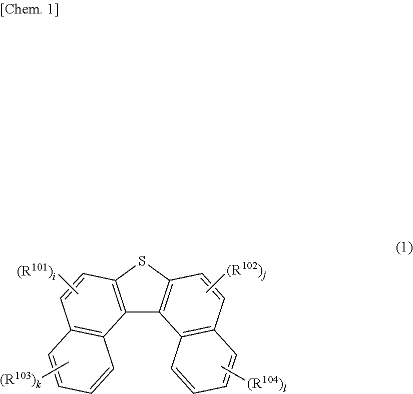
(In the general formula (1), R¹⁰¹ to R¹⁰⁴ are each independently a univalent substituent group represented by the following general formula (2-1), and i to 1 are each independently an integer of 0 or 1, provided that i to 1 are not simultaneously 0.) 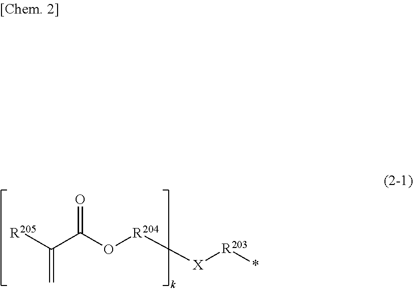
(In the general formula (2-1), R²⁰³ and R²⁰⁴ are each independently a single bond or a straight chain or branched substituted or unsubstituted alkylene group represented by CₙH₂ₙ (n is an integer of equal to or greater than 1), and R²⁰⁵ is hydrogen or a straight chain or branched substituted or unsubstituted alkyl group represented by CₙH₂ₙ₊₁ (n is an integer of equal to or greater than 1). Represented by k is an integer of equal to or greater than 1, and X is a bivalent or more-valent aromatic group. If carbon not bonded to R²⁰³ and R²⁰⁴ is present in the bivalent or more-valent aromatic group, the carbon is unsubstituted or has at least one substituent group. In addition, a part for bonding to R²⁰³ and at least one part for bonding to R²⁰⁴, possessed by the bivalent or more-valent aromatic group, may be any bondable carbon in the aromatic group. Represented by * in R¹⁰¹ to R¹⁰² is a part for bonding with carbon that is bondable in a benzene ring condensed with a thiophene ring in the general formula (1). Represented by * in R¹⁰³ to R¹⁰⁴ is a part for bonding with carbon that is bondable in the benzene ring not condensed with the thiophene ring in the general formula (1).)

2. The compound according to claim 1, wherein at least one carbon atom of at least one carbon skeleton, of carbon skeletons constituting the alkylene groups of R²⁰³ and R²⁰⁴ and the alkyl group of R²⁰⁵, is substituted by a hetero atom.

3. The compound according to claim 1, wherein at least one hydrogen atom, of hydrogen atoms constituting the alkylene group of R²⁰³, hydrogen atoms constituting the alkylene group of R²⁰⁴, and hydrogen atoms constituting the alkyl group of R²⁰⁵, is substituted by a halogen atom.

4. The compound according to claim 1, wherein R²⁰³ and R²⁰⁴ are single bonds or straight chain or branched substituted or unsubstituted alkylene groups represented by CₙH₂ₙ (n is an integer of 1≤n≤10), and R²⁰⁵ is hydrogen or a straight chain or branched substituted or unsubstituted alkyl group represented by CₙH₂ₙ₊₁ (n is an integer of 1≤n≤10).

5. The compound according to claim 4, wherein at least one carbon atom of at least one carbon skeleton, of carbon skeletons constituting the alkylene groups of R²⁰³ and R²⁰⁴ and the alkyl group of R²⁰⁵, is substituted by a hetero atom.

6. The compound according to claim 4, wherein at least one hydrogen atom, of hydrogen atoms constituting the alkylene group of R²⁰³, hydrogen atoms constituting the alkylene group of R²⁰⁴, and hydrogen atoms constituting the alkyl group of R²⁰⁵, is substituted by a halogen atom.

7. The compound according to claim 1, wherein X is a bivalent or more-valent aromatic group represented by the following general formulas (3-1) to (3-8).

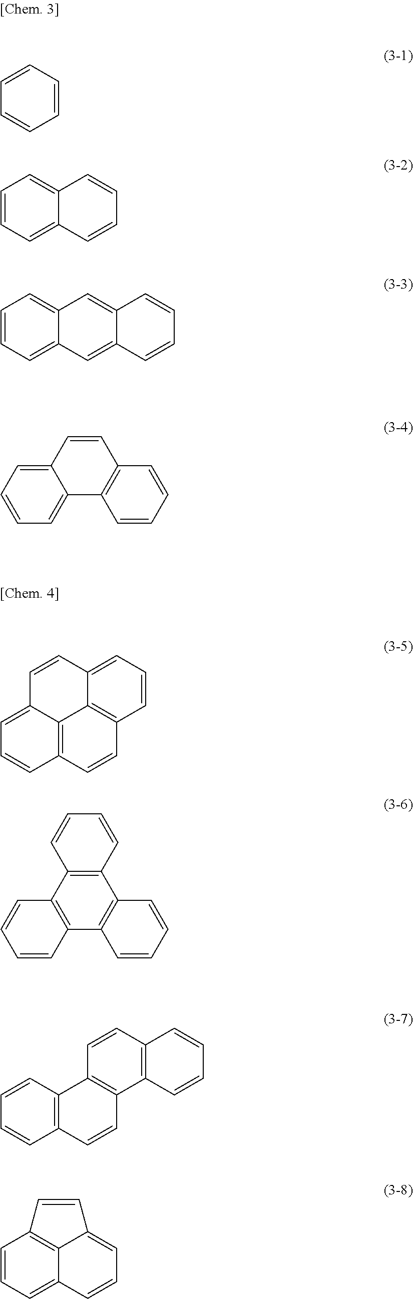

8. The compound according to claim 1, wherein k is 1, and X is a bivalent aromatic group.

9. The compound according to claim 8, wherein the bivalent aromatic group is a monocyclic arylene group, and two parts for bonding to R²⁰³ and R²⁰⁴, possessed by the monocyclic arylene group, are in a relation of ortho positions, meta positions, or para positions.

10. The compound according to claim 8, wherein the bivalent aromatic group is a polycyclic arylene group, and two parts for bonding to R²⁰³ and R²⁰⁴, possessed by the polycyclic arylene group, are any two bondable carbon atoms in the polycyclic arylene group.

11. The compound according to claim 1, wherein k is 2, and X is a trivalent aromatic group.

12. The compound according to claim 11, wherein the trivalent aromatic group is a monocyclic trivalent aromatic group, and two parts for bonding to R²⁰⁴, possessed by the monocyclic trivalent aromatic group, are in a relation of ortho positions, meta positions, or para positions.

13. The compound according to claim 1, wherein at least one of R¹⁰¹ or R¹⁰² is adjacent to a carbon atom adjacent to a sulfur atom in the general formula (1) and is bonded to bondable carbon in the benzene ring condensed with the thiophene ring in the general formula (1).

14. An organic material including the compound according to claim 1.

15. The organic material according to claim 14, that is an organic thin film, an organic lens, or a hologram.

16. The organic material according to claim 14, that is an organic thin film composition, an organic lens composition, or a hologram recording photosensitive composition.

17. A polymer obtained by polymerizing the compound according to claim 1.

18. An organic material including the polymer according to claim 17.

19. The organic material according to claim 18, that is an organic thin film, an organic lens, or a hologram.

20. The organic material according to claim 18, that is an organic thin film composition, an organic lens composition, or a hologram recording photosensitive composition.
